(19) United States
(12) Patent Application Publication
(10) Pub. No.: US 2004/0259819 A1
(43) Pub. Date: Dec. 23, 2004

(54) Novel heterocyclic fluoroglycoside derivatives, medicaments containing these compounds, and the use thereof

(75) Inventors: Wendelin Frick, Hunstetten-Beuerbach (DE); Heiner Glombik, Hofheim (DE); Werner Kramer, Mainz-Laubenheim (DE); Hubert Heuer, Schwabenheim (DE); Harm Brummerhop, Frankfurt (DE); Oliver Plettenburg, Hattersheim (DE)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 10/734,573

(22) Filed: Dec. 12, 2003

Related U.S. Application Data
(60) Provisional application No. 60/466,449, filed on Apr. 29, 2003.

(30) Foreign Application Priority Data
Dec. 12, 2002 (DE) 10258008.1-43
Nov. 28, 2003 (WO) PCT/EP03/13455

(51) Int. Cl. A61K 31/7056; C07H 19/056

(52) U.S. Cl. 514/43; 536/18.7; 536/28.7

(57) ABSTRACT

Novel heterocyclic fluoroglycoside derivatives, medicaments containing these compounds, and the use thereof.

The invention relates to substituted heterocyclic fluoroglycoside derivatives of the formula I

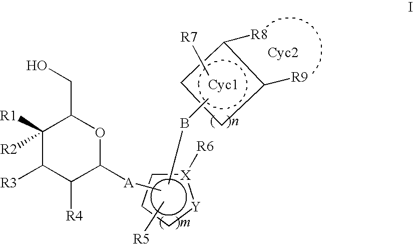

in which the radicals have the stated meanings, and their physiologically tolerated salts and processes for their preparation. The compounds are suitable for example as antidiabetics.

The invention relates to substituted heterocyclic fluoroglycoside derivatives, their physiologically tolerated salts and physiologically functional derivatives.

Several classes of substances having an SGLT effect have already been disclosed in the literature. The model for all these structures was the natural product phlorizin. From this were derived the following classes which are described in the property rights below:

propiophenone glycosides of Tanabe (WO 0280936, WO 0280935, JP 2000080041 and EP 850948)

2-(glucopyranosyloxy)benzylbenzenes of Kissei (WO 0244192, WO 0228872 and WO 0168660)

glucopyranosyloxypyrazoles of Kissei and Ajinomoto (WO 0268440, WO 0268439, WO 0236602 and WO 0116147)

O-glycoside benzamides of Bristol-Myers Squibb (WO 0174835 and WO 0174834)

and C-aryl glycosides of Bristol-Myers Squibb (WO 0127128 and US 2002137903).

All the known structures contain glucose as a very important structural element.

The invention was based on the object of providing novel compounds with which it is possible to prevent and treat type 1 and type 2 diabetes. We have now surprisingly found that heterocyclic fluoroglycoside derivatives increase the effect on SGLT. These compounds are therefore particularly suitable for preventing and treating type 1 and type 2 diabetes.

The invention therefore relates to compounds of the formula I

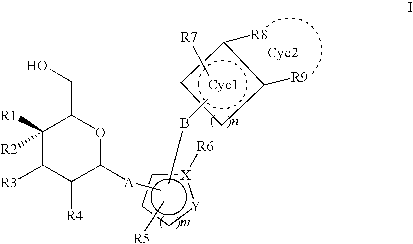

wherein

R1 and R2 are each independently F or H or one of said radicals R1 and R2 may be OH;

R3 is OH or F, with the proviso that at least one of the radicals R1, R2 and R3 must be F;

R4 is OH;

A is O, NH, CH₂, S or a bond;

X is C, O, S or N, with the proviso that X is C when Y is O or S;

Y is N, O or S;

m is 1 or 2;

R5 is hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl, (C₁-C₆)-alkoxycarboxyl,

wherein said CO(C₁-C₆)-alkyl, COO(C₁-C₆)-alkyl, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl and (C₁-C₆)-alkoxycarboxyl radicals are optionally substituted with one or more fluorine atoms,

SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl,

wherein said SO₂NH(C₁-C₆)-alkyl, SO₂N[(C₁-C₆)-alkyl]₂, S—(C₁-C₆)-alkyl, SO—(C₁-C₆)-alkyl and SO₂—(C₁-C₆)-alkyl radicals are optionally substituted with one or more fluorine atoms, and wherein the phenyl ring of said S—(CH₂)ₒ-phenyl, SO—(CH₂)ₒ-phenyl and SO₂—(CH₂)ₒ-phenyl radicals is optionally mono- or disubstituted with F, Cl, Br, OH, CF₃, NO₂, CN, OCF₃, O—(C₁-C₆)-alkyl, (C₁-C₆)-alkyl or NH₂, and wherein o is 0, 1, 2, 3, 4, 5, or 6,

NH₂, NH—(C₁-C₆)-alkyl, N((C₁-C₆)-alkyl)₂, NH(C₁-C₇)-acyl, phenyl or O—(CH₂)ₒ-phenyl,

wherein the phenyl ring of said phenyl and O—(CH₂)ₒ-phenyl radicals is optionally mono-, di-, or trisubstituted with F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂, and wherein o is as hereinabove defined;

or, when Y is S, R5 and R6 taken together with the carbon atoms to which they are attached may form a phenyl ring;

R6 is H, (C₁-C₆)-alkyl, (C₁-C₆)-alkenyl, (C₃-C₆)-cycloalkyl, or phenyl wherein said phenyl radical is optionally substituted with halogen or (C₁-C₄)-alkyl;

B is (C₀-C₁₅)-alkanediyl, wherein one or more of the carbon atoms in said alkanediyl radical may be replaced, independently of one another, with —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;

n is 0, 1, 2, 3 or 4;

Cyc1 is a 3-, 4-, 5-, 6- or 7-membered saturated, partially saturated or unsaturated ring, wherein one carbon atom of said ring may be replaced by O, N or S;

R7, R8, and R9 are each independently hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl,

wherein said COO(C₁-C₆)-alkyl, CO(C₁-C₄)-alkyl, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl and (C₁-C₆)-alkyl-O—(C₁-C₆)-alkyl radicals are optionally substituted with one or more fluorine atoms,

SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SCF₃, SO—(C₁-C₆)-alkyl, SO—(CH₂)ₒ-phenyl, SO₂—(C₁-C₆)-alkyl, SO₂—(CH₂)ₒ-phenyl,

wherein said SO₂NH(C₁-C₆)-alkyl, SO₂N[(C₁-C₆)-alkyl]₂, S—(C₁-C₆)-alkyl, SO—(C₁-C₆)-alkyl and SO₂—(C₁-C₆)-alkyl radicals are optionally substituted with one or more fluorine atoms, and wherein the phenyl ring of said S—(CH₂)ₒ-phenyl, SO—(CH₂)ₒ-phenyl and SO₂—(CH₂)ₒ-phenyl radicals is optionally mono- or disubstituted with F, Cl, Br, OH, CF₃, NO₂, CN, OCF₃, O—(C₁-C₆)-alkyl, (C₁-C₆)-alkyl or NH₂, and wherein o is as hereinabove defined,

NH₂, NH—(C₁-C₆)-alkyl, N((C₁-C₆)-alkyl)₂, NH(C₁-C₇)-acyl, phenyl or O—(CH₂)ₒ-phenyl,

wherein the phenyl ring of said phenyl and O—(CH₂)ₒ-phenyl radicals is optionally mono-, di-, or trisubstituted with F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂, and wherein o is as hereinabove defined;

or R8 and R9 taken together with the carbon atoms to which they are attached form a 5-, 6- or 7-membered, saturated, partially saturated or completely unsaturated ring herein referred to as Cyc2,

wherein one or two carbon atom(s) in said Cyc2 ring are optionally replaced by N, O or S, and wherein said Cyc2 ring is optionally substituted with (C₁-C₆)-alkyl, (C₂-C₅)— alkenyl or (C₂-C₅)-alkynyl,

wherein said (C₁-C₆)-alkyl, (C₂-C₅)-alkenyl and (C₂-C₅)-alkynyl radicals are optionally substituted with F, Cl, OH, CF₃, NO₂, CN, COO(C₁-C₄)-alkyl, CONH₂, CONH(C₁-C₄)-alkyl or OCF₃, and wherein a —CH₂— group contained in said (C₁-C₆)-alkyl, (C₂-C₅)-alkenyl and (C₂-C₅)-alkynyl radicals is optionally replaced by —O—;

and pharmaceutically acceptable salts thereof.

The points of linkage of A, B and R₅ to the ring can be chosen without restriction. The present invention includes all the resulting compounds of the formula I.

Suitable heterocycles of the central building block comprising X and Y are: thiophene, furan, pyrrole, pyrazole, isoxazole and isothiazole, with preference for thiophene, pyrazole and isoxazole. Particularly preferred compounds of the formula I are those comprising thiophene or pyrazole as central building block.

Preferred compounds of the formula I are those wherein:

R1 and R2 are each independently F or H or one of said radicals R1 and R2 may be OH,

with the proviso that at least one of said radicals R1 and R2 is F;

R3 is OH;

R4 is OH;

A is O or NH;

X is C, O or N, with the proviso that X is C when Y is S;

Y is N or S;

m is 1 or 2;

R5 is hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl or (C1-C6)-alkoxycarboxyl,

wherein said CO(C₁-C₆)-alkyl, COO(C₁-C₆)-alkyl, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, (C₁-C₆)-alkoxycarboxyl and SO—(C₁-C₆)-alkyl radicals are optionally substituted with one or more fluorine atoms,

or when Y is S, R5 and R6 taken together with the carbon atoms to which they are attached may form a phenyl ring;

R6 is H, (C₁-C₆)-alkyl, (C₁-C₆)-alkenyl, (C₃-C₆)-cycloalkyl, or phenyl wherein said phenyl radical is optionally substituted with halogen or (C₁-C₄)-alkyl;

B is (C₀-C₁₅)-alkanediyl, wherein one or more of the carbon atoms in said alkanediyl radical may be replaced, independently of one another, with —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;

n is 0, 1, 2, 3 or 4;

Cyc1 is a 3-, 4-, 5-, 6- or 7-membered saturated, partially saturated or unsaturated ring, wherein one carbon atom of said ring may be replaced by O or S;

R7, R8, and R9 are each independently hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, S—(C₁-C₆)-alkyl, CF₃ or SO—(C₁-C₆)-alkyl,

wherein said COO(C₁-C₆)-alkyl, CO(C₁-C₄)-alkyl, CON H(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, S—(C₁-C₆)-alkyl and SO—(C₁-C₆)-alkyl radicals are optionally substituted with one or more fluorine atoms,

or R8 and R9 taken together with the carbon atoms to which they are attached form a 5-, 6- or 7-membered, saturated, partially saturated or completely unsaturated ring herein referred to as Cyc2,

wherein one or two carbon atom(s) in said Cyc2 ring is optionally replaced by N, O or S, and wherein said Cyc2 ring is optionally substituted with (C₁-C₆)-alkyl, (C₂-C₅)-alkenyl or (C₂-C₅)-alkynyl,

wherein said (C₁-C₆)-alkyl, (C₂-C₅)-alkenyl and (C₂-C₅)-alkynyl radicals are optionally substituted with F, Cl, OH, CF₃, NO₂, CN, COO(C₁-C₄)-alkyl, CONH₂, CONH(C₁-C₄)-alkyl or OCF₃, and wherein a —CH₂— group contained in said (C₁-C₆)-alkyl, (C₂-C₅)-alkenyl and (C₂-C₅)-alkynyl radicals is optionally replaced by —O—.

Further preferred compounds of the formula I are those in which the sugar residues are beta(β)-linked and the stereochemistry in the 2, 3 and 5 position of the sugar residue has the D-gluco configuration.

Particularly preferred compounds of the formula I are those in which the substituents A and B occupy an adjacent position (ortho position).

Particularly preferred compounds of the formula I wherein:

R1 and R2 are each independently F or H or one of said radicals R1 and R2 may be OH,

with the proviso that at least one of said radicals R1 and R2 is F;

R3 is OH;

R4 is OH;

A is O;

X is C, O or N, with the proviso that X is C when Y is S;

Y is N or S;

m is 1;

R5 is hydrogen, F, Cl, CF₃, OCF₃, COO(C₁-C₄)-alkyl, (C₁-C₅)-alkyl, (C₂-C₄)-alkenyl, (C₂-C₄)-alkynyl, (C₁-C₄)-alkoxy, HO—(C₁-C₄)-alkyl, (C₁-C₄)-alkyl-O—(C₁-C₄)-alkyl, phenyl, benzyl, (C₁-C₄)-alkoxycarboxyl, OCH₂CF₃ or (C₁-C₄)-alkyl-CF₂—,

or when Y is S, R5 and R6 taken together with the carbon atoms to which they are attached may form a phenyl ring;

R6 is H, (C₁-C₆)-alkyl, (C₁-C₆)-alkenyl, (C₃-C₆)-cycloalkyl, or phenyl wherein said phenyl radical is optionally substituted with halogen or (C₁-C₄)-alkyl;

B is (C₁-C₄)-alkanediyl, wherein one carbon atom in said alkanediyl radical may be replaced with —O—, —(C═O)—, —CH(OH)—, —CHF—, —CF₂—, —CO—NH—;

n is 2 or 3;

Cyc1 is an unsaturated 5- or 6-membered ring, wherein one carbon atom of said ring may be replaced by O or S;

R7, R8, and R9 are each independently hydrogen, F, Cl, Br, I, OH, (C₁-C₄)-alkyl, OCH₂CF₃, (C₁-C₈)-alkoxy, HO—(C₁-C₆)-alkyl, (C₁-C₄)-alkyl-O—(C₁-C₄)-alkyl, S—(C₁-C₄)-alkyl, SCF₃ or OCF₃,

or R8 and R9 taken together form the radicals —C═CH—O—, —CH═CH—S— or —CH═CH—CH═CH— and, with the carbon atoms to which they are attached, form an unsaturated or partially saturated 5- or 6-membered ring, said ring being optionally substituted by (C₁-C₄)-alkoxy or —O—(CH₂)ₚ—O— wherein p is 1 or 2 and, in such instance, R7 is preferably hydrogen.

Very particularly preferred compounds of the formula I are those wherein:

R1 and R2 are each independently F or H, with the proviso that at least one of said radicals R1 and R2 is F;

R3 is OH;

R4 is OH;

A is O;

X is C and Y is S, or

is O and Y is N, or

is N and Y is N;

m is 1;

R5 is hydrogen, CF₃, (C₁-C₆)-alkyl, or when Y is S, R5 and R6 taken together with the carbon atoms to which they are attached may form a phenyl ring,

R6 is H, (C₁-C₄)-alkyl or phenyl;

B is —CH₂—, —C2H₄—, —C₃H₆—, —CO—NH—CH₂— or —CO—CH₂—CH₂—;

n is 2 or 3;

Cyc1 is an unsaturated 5- or 6-membered ring, wherein one carbon atom of said ring may be replaced by S;

R7, R8, and R9 are each independently hydrogen, F, Cl, Br, I, (C₁-C₆)-alkyl, (C₁-C₄)-alkoxy, S—(C₁-C₄)-alkyl, SCF3 or OCF3,

or R8 and R9 taken together form the radicals —C═CH—O— or —CH═CH—CH═CH— and, with the carbon atoms to which they are attached, form an unsaturated or partially saturated 5- or 6-membered ring, said ring being optionally substituted by (C₁-C₄)-alkoxy, and, in such instance R₇ is preferably hydrogen.

Further very particularly preferred compounds of the formula I are those wherein:

R1 and R2 are each independently F or H,

with the proviso that at least one of said radicals R1 and R2 is F;

R3 is OH;

R4 is OH;

A is O;

X is C and Y is S, or

is N and Y is N;

m is 1;

R5 is hydrogen, CF₃, (C₁-C₆)-alkyl, or when Y is S, R5 and R6 taken together with the carbon atoms to which they are attached may form a phenyl ring,

R6 is H or (C₁-C₄)-alkyl;

B is —CH₂— or —CO—NH—CH₂—;

n is 2 or 3;

Cyc1 is phenyl or thiophene;

R7, R8, and R9 are each independently hydrogen or Cl,

or R8 and R9 taken together with the carbon atoms to which they are attached, form a furan ring or a phenyl ring optionally substituted with methoxy, and, in such instance, R7 is preferably hydrogen.

The linkage of one of the substituents A or B particularly preferably takes place in a position adjacent to the variable Y.

Additional very particularly preferred compounds which may be mentioned are those in which Y is S and those in which R1 is H and R2 is F.

The invention relates to compounds of the formula I in the form of their racemates, racemic mixtures and pure enantiomers and to their diastereomers and mixtures thereof.

The alkyl radicals in the substituents R4, R5, R6, R7, R8 and R9 may be either straight-chain or branched. Halogen means F, Cl, Br, I, preferably F or Cl.

Pharmaceutically acceptable salts are, because their solubility in water is greater than that of the initial or basic compounds, particularly suitable for medical applications. These salts must have a pharmaceutically acceptable anion or cation. Suitable pharmaceutically acceptable acid addition salts of the compounds of the invention are salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. Suitable pharmaceutically acceptable basic salts are ammonium salts, alkali metal salts (such as sodium and potassium salts), alkaline earth metal salts (such as magnesium and calcium salts), and salts of trometamol (2-amino-2-hydroxymethyl-1,3-propanediol), diethanolamine, lysine or ethylenediamine.

Salts with a pharmaceutically unacceptable anion such as, for example, trifluoroacetate likewise belong within the framework of the invention as useful intermediates for the preparation or purification of pharmaceutically acceptable salts and/or for use in nontherapeutic, for example in vitro, applications.

The term “physiologically functional derivative” used herein refers to any physiologically tolerated derivative of a compound of the formula I of the invention, for example an ester, which on administration to a mammal such as, for example, a human is able to form (directly or indirectly) a compound of the formula I or an active metabolite thereof.

Physiologically functional derivatives include prodrugs of the compounds of the invention, as described, for example, in H. Okada et al., Chem. Pharm. Bull. 1994, 42, 57-61. Such prodrugs can be metabolized in vivo to a compound of the invention. These prodrugs may themselves be active or not.

The compounds of the invention may also exist in various polymorphous forms, for example as amorphous and crystalline polymorphous forms. All polymorphous forms of the compounds of the invention belong within the framework of the invention and are a further aspect of the invention.

All references to “compound(s) of formula I” hereinafter refer to compound(s) of the formula I as described above, and their salts, solvates and physiologically functional derivatives as described herein.

“Patient” means a warm blooded animal, such as for example rat, mice, dogs, cats, guinea pigs, and primates such as humans.

“Treat” or “treating” means to alleviate symptoms, eliminate the causation of the symptoms either on a temporary or permanent basis, or to prevent or slow the appearance of symptoms of the named disorder or condition.

“Therapeutically effective amount” means a quantity of the compound which is effective in treating the named disorder or condition.

“Pharmaceutically acceptable carrier” is a non-toxic solvent, dispersant, excipient, adjuvant or other material which is mixed with the active ingredient in order to permit the formation of a pharmaceutical composition, i.e., a dosage form capable of administration to the patient. One example of such a carrier is a pharmaceutically acceptable oil typically used for parenteral administration.

The compound(s) of formula (I) may also be administered in combination with other active ingredients.

The amount of a compound of formula I necessary to achieve the desired biological effect depends on a number of factors, for example the specific compound chosen, the intended use, the mode of administration and the clinical condition of the patient. The daily dose is generally in the range from 0.3 mg to 100 mg (typically from 3 mg to 50 mg) per day and per kilogram of bodyweight, for example 3-10 mg/kg/day. An intravenous dose may be, for example, in the range from 0.3 mg to 1.0 mg/kg, which can suitably be administered as infusion of 10 ng to 100 ng per kilogram and per minute. Suitable infusion solutions for these purposes may contain, for example, from 0.1 ng to 10 mg, typically from 1 ng to 10 mg, per milliliter. Single doses may contain, for example, from 1 mg to 10 g of the active ingredient. Thus, ampoules for injections may contain, for example, from 1 mg to 100 mg, and single-dose formulations which can be administered orally, such as, for example, tablets or capsules, may contain, for example, from 1.0 to 1000 mg, typically from 10 to 600 mg. For the therapy of the above-mentioned conditions, the compounds of formula I may be used as the compound itself, but they are preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of formula I. The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral, rectal, topical, peroral (for example sublingual) and parenteral (for example subcutaneous, intramuscular, intradermal or intravenous) administration, although the most suitable mode of administration depends in each individual case on the nature and severity of the condition to be treated and on the nature of the compound of formula I used in each case. Coated formulations and coated slow-release formulations also belong within the framework of the invention. Preference is given to acid- and gastric juice-resistant formulations. Suitable coatings resistant to gastric juice comprise cellulose acetate phthalate, polyvinyl acetate phthalate, hydroxypropylmethylcellulose phthalate and anionic polymers of methacrylic acid and methyl methacrylate.

Suitable pharmaceutical compounds for oral administration may be in the form of separate units such as, for example, capsules, cachets, suckable tablets or tablets, each of which contain a defined amount of the compound of formula I; as powders or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary. Thus, for example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent and/or one (or more) surface-active/dispersing agent(s) in a suitable machine. Molded tablets can be produced by molding the compound, which is in powder form and is moistened with an inert liquid diluent, in a suitable machine.

Pharmaceutical compositions which are suitable for peroral (sublingual) administration comprise suckable tablets which contain a compound of formula I with a flavoring, normally sucrose and gum arabic or tragacanth, and pastilles which comprise the compound in an inert base such as gelatin and glycerol or sucrose and gum arabic.

Pharmaceutical compositions suitable for parenteral administration comprise preferably sterile aqueous preparations of a compound of formula I, which are preferably isotonic with the blood of the intended recipient. These preparations are preferably administered intravenously, although administration may also take place by subcutaneous, intramuscular or intradermal injection. These preparations can preferably be produced by mixing the compound with water and making the resulting solution sterile and isotonic with blood. Injectable compositions of the invention generally contain from 0.1 to 5% by weight of the active compound.

Pharmaceutical compositions suitable for rectal administration are preferably in the form of single-dose suppositories. These can be produced by mixing a compound of formula I with one or more conventional solid carriers, for example cocoa butter, and shaping the resulting mixture.

Pharmaceutical compositions suitable for topical use on the skin are preferably in the form of ointment, cream, lotion, paste, spray, aerosol or oil. Carriers which can be used are petrolatum, lanolin, polyethylene glycols, alcohols and combinations of two or more of these substances. The active ingredient is generally present in a concentration of from 0.1 to 15% by weight of the composition, for example from 0.5 to 2%.

Transdermal administration is also possible. Pharmaceutical compositions suitable for transdermal uses can be in the form of single plasters which are suitable for long-term close contact with the patient's epidermis. Such plasters suitably contain the active ingredient in an aqueous solution which is buffered where appropriate, dissolved and/or dispersed in an adhesive or dispersed in a polymer. A suitable active ingredient concentration is about 1% to 35%, preferably about 3% to 15%. A particular possibility is for the active ingredient to be released by electrotransport or iontophoresis as described, for example, in Pharmaceutical Research, 2(6): 318 (1986).

The invention also relates to processes for preparing the compounds of the formula I, which can be obtained as shown in the following reaction schemes for processes A, B and C;

Process A:

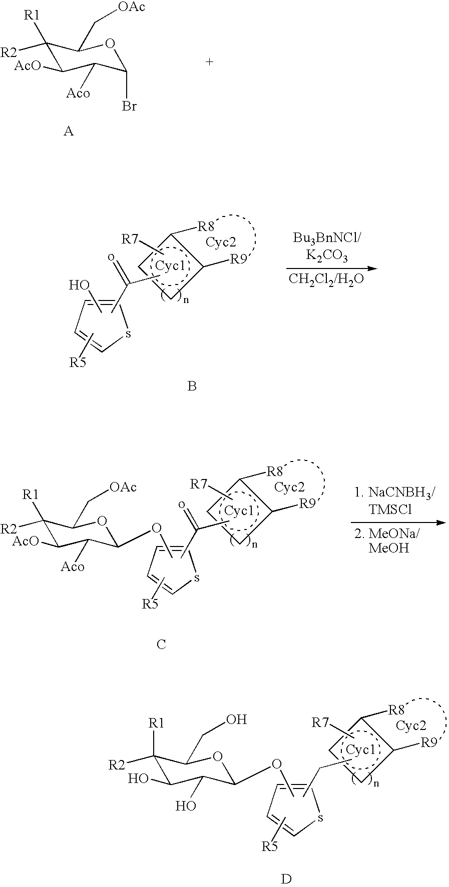

Process B:

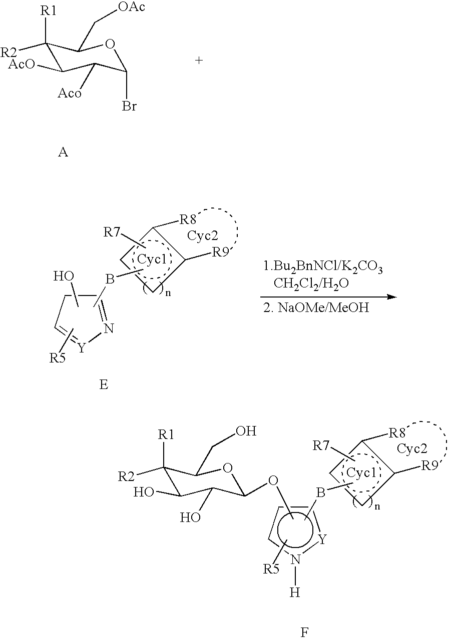

Process C:

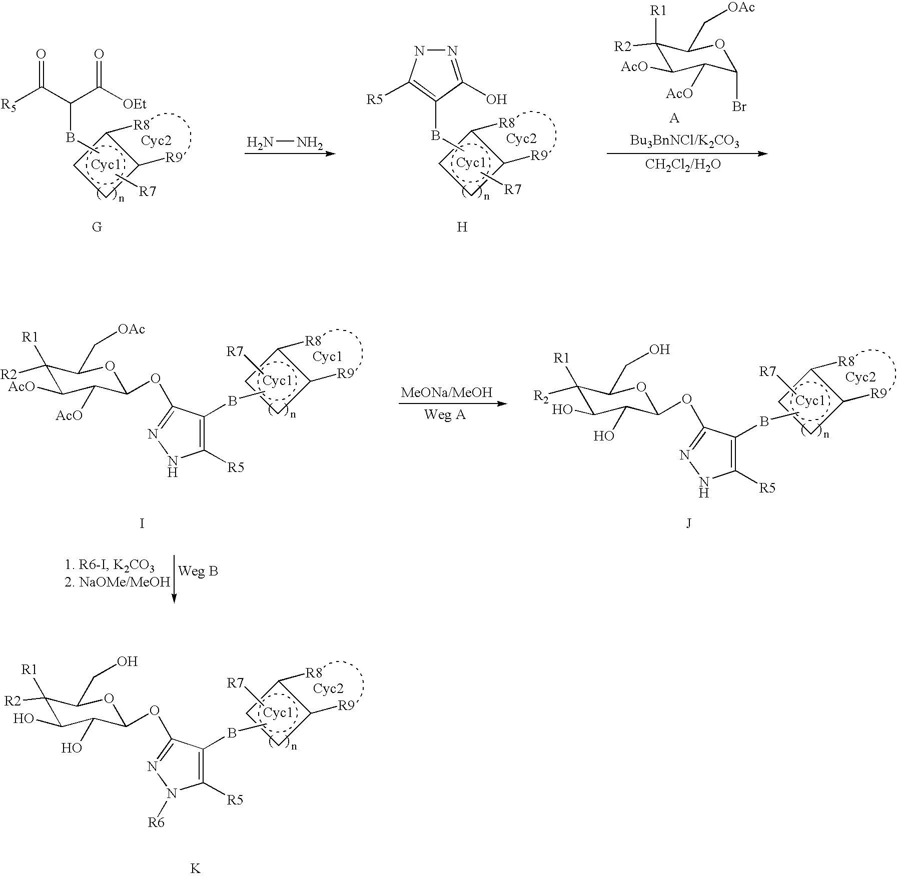

The schemes depicted for processes A, B and C are self-explanatory and can be carried out thus by the skilled worker. More details are, nevertheless, indicated in the experimental part. The compounds of examples 1 to 31 were obtained by processes A, B and C. Other compounds of the formula I can be obtained correspondingly or by known processes.

The compound(s) of the formula I can also be administered in combination with other active ingredients.

Further active ingredients suitable for combination products are:

all antidiabetics mentioned in the Rote Liste 2001, chapter 12. They may be combined with the compounds of the formula I of the invention in particular for synergistic improvement of the effect. Administration of the active ingredient combination may take place either by separate administration of the active ingredients to the patient or in the form of combination products in which a plurality of active ingredients are present in one pharmaceutical preparation. Most of the active ingredients listed below are disclosed in the USP Dictionary of USAN and International Drug Names, US Pharmacopeia, Rockville 2001.

Antidiabetics include insulin and insulin derivatives such as, for example, Lantus® (see www.lantus.com) or HMR 1964, fast-acting insulins (see U.S. Pat. No. 6,221,633), GLP-1 derivatives such as, for example, those disclosed in WO 98/08871 of Novo Nordisk A/S, and orally effective hypoglycemic active ingredients.

The orally effective hypoglycemic active ingredients include, preferably, sulfonylureas, biguanides, meglitinides, oxadiazolidinediones, thiazolidinediones, glucosidase inhibitors, glucagon antagonists, GLP-1 agonists, potassium channel openers such as, for example, those disclosed in WO 97/26265 and WO 99/03861 of Novo Nordisk A/S, insulin sensitizers, inhibitors of liver enzymes involved in the stimulation of gluconeogenesis and/or glycogenolysis, modulators of glucose uptake, compounds which alter lipid metabolism, such as antihyperlipidemic active ingredients and antilipidemic active ingredients, compounds which reduce food intake, PPAR and PXR agonists and active ingredients which act on the ATP-dependent potassium channel of the beta cells.

In one embodiment of the invention, the compounds of the formula I are administered in combination with an HMGCOA reductase inhibitor such as simvastatin, fluvastatin, pravastatin, lovastatin, atorvastatin, cerivastatin, rosuvastatin.

In one embodiment of the invention, the compounds of the formula I are administered in combination with a cholesterol absorption inhibitor such as, for example, ezetimibe, tiqueside, pamaqueside.

In one embodiment of the invention, the compounds of the formula I are administered in combination with a PPAR gamma agonist, such as, for example, rosiglitazone, pioglitazone, JTT-501, GI 262570.

In one embodiment of the invention, the compounds of the formula I are administered in combination with a PPAR alpha agonist, such as, for example, GW 9578, GW 7647.

In one embodiment of the invention, the compounds of the formula I are administered in combination with a mixed PPAR alpha/gamma agonist, such as, for example, GW 1536, AVE 8042, AVE 8134, AVE 0847, AVE 0897 or as described in WO 00/64888, WO 00/64876, WO 03/20269.

In one embodiment of the invention, the compounds of the formula I are administered in combination with a fibrate such as, for example, fenofibrate, clofibrate, bezafibrate.

In one embodiment of the invention, the compounds of the formula I are administered in combination with an MTP inhibitor such as, for example, implitapide, BMS-201038, R-103757.

In one embodiment of the invention, the compounds of the formula I are administered in combination with bile acid absorption inhibitor (see, for example, U.S. Pat. No. 6,245,744 or U.S. Pat. No. 6,221,897), such as, for example, HMR 1741.

In one embodiment of the invention, the compounds of the formula I are administered in combination with a CETP inhibitor, such as, for example, JTT-705.

In one embodiment of the invention, the compounds of the formula I are administered in combination with a polymeric bile acid adsorbent such as, for example, cholestyramine, colesevelam.

In one embodiment of the invention, the compounds of the formula I are administered in combination with an LDL receptor inducer (see U.S. Pat. No. 6,342,512), such as, for example, HMR1171, HMR1586.

In one embodiment of the invention, the compounds of the formula I are administered in combination with an ACAT inhibitor, such as, for example, avasimibe.

In one embodiment of the invention, the compounds of the formula I are administered in combination with an antioxidant, such as, for example, OPC-14117.

In one embodiment of the invention, the compounds of the formula I are administered in combination with a lipoprotein lipase inhibitor, such as, for example, NO-1886.

In one embodiment of the invention, the compounds of the formula I are administered in combination with an ATP-citrate lyase inhibitor, such as, for example, SB-204990.

In one embodiment of the invention, the compounds of the formula I are administered in combination with a squalene synthetase inhibitor, such as, for example, BMS-188494.

In one embodiment of the invention, the compounds of the formula I are administered in combination with a lipoprotein(a) antagonist, such as, for example, Cl-1027 or nicotinic acid.

In one embodiment of the invention, the compounds of the formula I are administered in combination with a lipase inhibitor, such as, for example, orlistat.

In one embodiment of the invention, the compounds of the formula I are administered in combination with insulin.

In one embodiment, the compounds of the formula I are administered in combination with a sulfonylurea such as, for example, tolbutamide, glibenclamide, glipizide or glimepiride.

In one embodiment, the compounds of the formula I are administered in combination with a biguanide, such as, for example, metformin.

In one further embodiment, the compounds of the formula I are administered in combination with a meglitinide, such as, for example, repaglinide.

In one embodiment, the compounds of the formula I are administered in combination with a thiazolidinedione, such as, for example, troglitazone, ciglitazone, pioglitazone, rosiglitazone or the compounds disclosed in WO 97/41097 of Dr. Reddy's Research Foundation, in particular 5-[[4-[(3,4-dihydro-3-methyl-4-oxo-2-quinazolinylmethoxy]phenyl]methyl]-2,4-thiazolidinedione.

In one embodiment, the compounds of the formula I are administered in combination with an a-glucosidase inhibitor, such as, for example, miglitol or acarbose.

In one embodiment, the compounds of the formula I are administered in combination with an active ingredient which acts on the ATP-dependent potassium channel of the beta cells, such as, for example, tolbutamide, glibenclamide, glipizide, glimepiride or repaglinide.

In one embodiment, the compounds of the formula I are administered in combination with more than one of the aforementioned compounds, e.g. in combination with a sulfonylurea and mefformin, with a sulfonylurea and acarbose, repaglinide and mefformin, insulin and a sulfonylurea, insulin and mefformin, insulin and troglitazone, insulin and lovastatin, etc.

In a further embodiment, the compounds of the formula I are administered in combination with CART modulators (see “Cocaine-amphetamine-regulated transcript influences energy metabolism, anxiety and gastric emptying in mice” Asakawa, A, et al., M.: Hormone and Metabolic Research (2001), 33(9), 554-558), NPY antagonists, e.g. naphthalene-1-sulfonic acid {4-[(4-aminoquinazolin-2-ylamino)methyl]-cyclohexylmethyl}amide; hydrochloride (CGP 71683A)), MC4 agonists (e.g. 1-amino-1,2,3,4-tetrahydronaphthalene-2-carboxylic acid [2-(3a-benzyl-2-methyl-3-oxo-2,3,3a,4,6,7-hexahydropyrazolo[4,3-c]pyridin-5-yl)-1-(4-chlorophenyl)-2-oxoethyl]-amide; (WO 01/91752)), orexin antagonists (e.g. 1-(2-methylbenzoxazol-6-yl)-3-[1,5]naphthyridin-4-ylurea; hydrochloride (SB-334867-A)), H3 agonists (3-cyclohexyl-1-(4,4-dimethyl-1,4,6,7-tetrahydroimidazo[4,5-c]pyridin-5-yl)propan-1-one oxalic acid salt (WO 00/63208)); TNF agonists, CRF antagonists (e.g. [2-methyl-9-(2,4,6-trimethyl phenyl)-9H-1,3,9-triazafluoren-4-yl]dipropylamine (WO 00/66585)), CRF BP antagonists (e.g. urocortin), urocortin agonists, β3 agonists (e.g. 1-(4-chloro-3-methanesulfonylmethyl phenyl)-2-[2-(2,3-dimethyl-1H-indol-6-yloxy)ethylamino]-ethanol; hydrochloride (WO 01/83451)), MSH (melanocyte-stimulating hormone) agonists, CCK-A agonists (e.g. {2-[4-(4-chloro-2,5-dimethoxyphenyl)-5-(2-cyclohexylethyl)thiazol-2-yl-carbamoyl]-5,7-dimethylindol-1-yl}acetic acid trifluoroacetic acid salt (WO 99/15525)), serotonin reuptake inhibitors (e.g. dexfenfluramine), mixed serotoninergic and noradrenergic compounds (e.g. WO 00/71549), 5HT agonists, e.g. 1-(3-ethylbenzofuran-7-yl)piperazine oxalic acid salt (WO 01/09111), bombesin agonists, galanin antagonists, growth hormone (e.g. human growth hormone), growth hormone-releasing compounds (6-benzyloxy-1-(2-diisopropylaminoethylcarbamoyl)-3,4-dihydro-1H-isoquinoline-2-carboxylic acid tert-butyl ester (WO 01/85695)), TRH agonists (see, for example, EP 0 462 884), uncoupling protein 2 or 3 modulators, leptin agonists (see, for example, Lee, Daniel W.; Leinung, Matthew C.; Rozhavskaya-Arena, Marina; Grasso, Patricia. Leptin agonists as a potential approach to the treatment of obesity. Drugs of the Future (2001), 26(9), 873-881), DA agonists (bromocriptine, Doprexin), lipase/amylase inhibitors (e.g. WO 00/40569), PPAR modulators (e.g. WO 00/78312), RXR modulators or TR-β agonists.

In one embodiment of the invention, the other active ingredient is leptin; see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In one embodiment, the other active ingredient is dexamphetamine or amphetamine.

In one embodiment, the other active ingredient is fenfluramine or dexfenfluramine.

In another embodiment, the other active ingredient is sibutramine.

In one embodiment, the other active ingredient is orlistat.

In one embodiment, the other active ingredient is mazindol or phentermine.

In one embodiment, the compounds of the formula I are administered in combination with bulking agents, preferably insoluble bulking agents (see, for example, carob/Caromaxe (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Höchst, 65926 Frankfurt/Main)). Combination with Caromax® is possible in one preparation or by separate administration of compounds of the formula I and Caromax®. Caromax® can in this connection also be administered in the form of food products such as, for example, in bakery products or muesli bars.

It will be appreciated that every suitable combination of the compounds of the invention with one or more of the aforementioned compounds and optionally one or more other pharmacologically active substances is regarded as falling within the protection conferred by the present invention.

The citation of any reference herein should not be construed as an admission that such reference is available as “Prior Art” to the instant application.

Various publications are cited herein, the disclosures of which are incorporated by reference in their entireties.

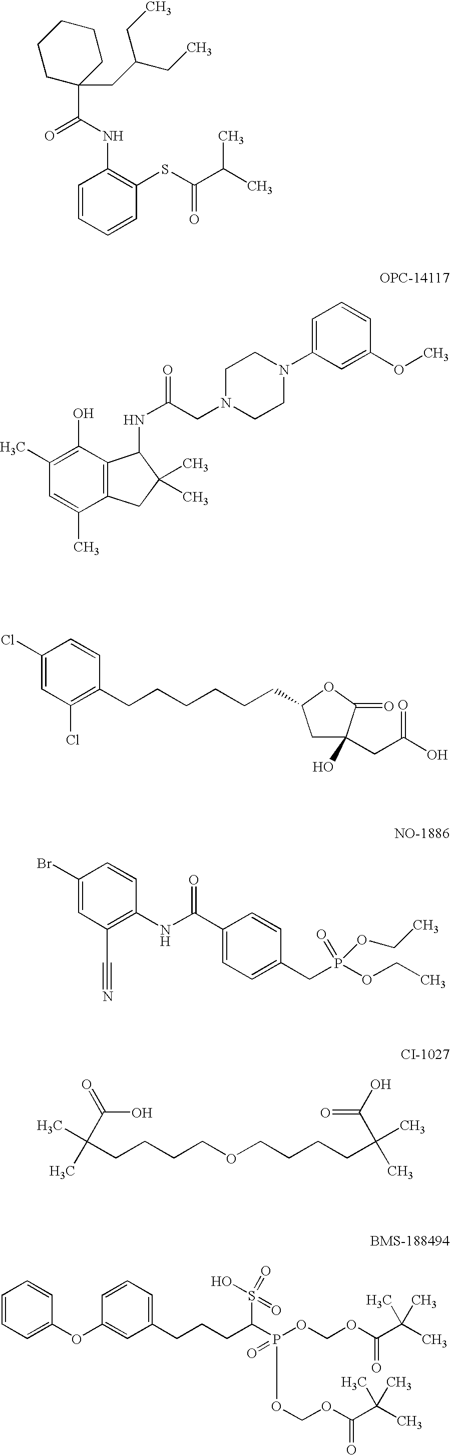

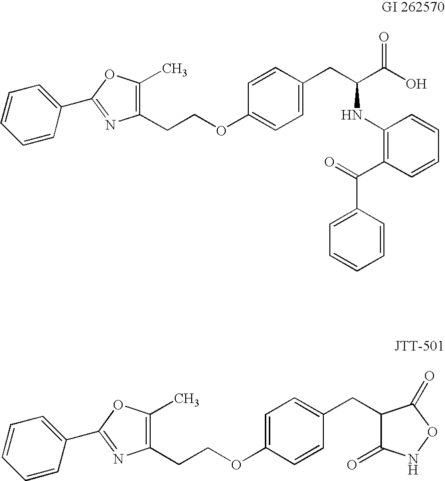

The examples detailed below serve to illustrate the invention without, however, restricting it.

TABLE 1
Compounds of the formula I

I

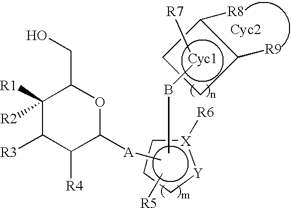

| Ex. | R1 | R2 | R3 | R4 | R5 | R6 | R7 | R8, R9 | A |
|---|---|---|---|---|---|---|---|---|---|
| 1 | H | F | OH | OH | H | — | 4-O—CH₃ | H, H | O |
| 2 | H | F | OH | OH | —CH═CH—CH═CH— | | 4-O—CH₃ | H, H | O |
| 3 | F | H | OH | OH | O | — | 4-O—CH₃ | H, H | O |
| 4 | H | OH | F | OH | OH | — | 4-O—CH₃ | H, H | O |
| 5 | H | F | OH | OH | CF₃ | H | 4-O—CH₃ | H, H | O |
| 6 | F | H | OH | OH | CF₃ | H | 4-O—CH₃ | H, H | O |
| 7 | H | F | OH | OH | CH₃ | H | 4-F | H, H | O |
| 8 | H | F | OH | OH | CH₃ | H | 2-Cl | 4-Cl, H | O |
| 9 | H | F | OH | OH | CH₃ | CH₃ | 4-F | H, H | O |
| 10 | H | F | OH | OH | CH₃ | CH₃ | 2-Cl | 4-Cl, H | O |
| 11 | H | F | OH | OH | H | — | 4-CH₂—CH₃ | H, H | O |
| 12 | H | OH | F | OH | H | — | 4-CH₂—CH₃ | H, H | O |
| 13 | H | F | OH | OH | H | — | 4-O—CH₃ | H, H | O |
| 14 | H | F | OH | OH | H | — | 4-O—CF₃ | H, H | O |
| 15 | H | F | OH | OH | CH₃ | — | 4-O—CH₃ | H, H | O |
| 16 | H | F | OH | OH | H | | H | —CH═CH—CH═CH— | O |
| 17 | H | F | OH | OH | H | — | 4-CH₃ | H, H | O |
| 18 | H | F | OH | OH | H | — | 2-CH₃ | H, H | O |
| 19 | H | F | OH | OH | H | — | 4-I | H, H | O |
| 20 | F | F | OH | OH | CF₃ | H | 4-O—CH₃ | H, H | O |
| 21 | H | F | OH | OH | H | — | 3-Me | H, H | O |
| 22 | H | F | OH | OH | H | — | 4-Cl | H, H | O |
| 23 | H | F | OH | OH | H | — | 4-F | H, H | O |
| 24 | H | F | OH | OH | H | — | H | —CH═CH—CH═CH— | O |
| 25 | H | F | OH | OH | H | — | 4-OCF₃ | H, H | O |
| 26 | H | F | OH | OH | H | — | 4-Br | H, H | O |
| 27 | H | F | OH | OH | H | — | 4-CH(CH₃)₂ | H, H | O |
| 28 | H | F | OH | OH | H | — | H | —CH═CH—C(OMe)═CH— | O |
| 29 | H | F | OH | OH | H | — | H | —CH═CH—O— | O |
| 30 | H | F | OH | OH | CH₃ | H | 2-F | H, H | O |
| 31 | H | F | OH | OH | CH₃ | H | 4-Cl | H, H | O |

| Ex. | B | Cyc1 | X | Y | m | n | MS* |
|---|---|---|---|---|---|---|---|
| 1 | CH₂ | Ph | C | S | 1 | 3 | ok |
| 2 | CH₂ | Ph | C | S | 1 | 3 | ok |
| 3 | CH₂ | Ph | C | S | 1 | 3 | ok |
| 4 | CH₂ | Ph | C | S | 1 | 3 | ok |
| 5 | CH₂ | Ph | N | N | 1 | 3 | ok |
| 6 | CH₂ | Ph | N | N | 1 | 3 | ok |
| 7 | CH₂ | Ph | N | N | 1 | 3 | ok |
| 8 | CH₂ | Ph | N | N | 1 | 3 | ok |
| 9 | CH₂ | Ph | N | N | 1 | 3 | ok |
| 10 | CH₂ | Ph | N | N | 1 | 3 | ok |
| 11 | CH₂ | Ph | C | S | 1 | 3 | ok |
| 12 | CH₂ | Ph | C | S | 1 | 3 | ok |
| 13 | CONHCH₂ | Ph | C | S | 1 | 3 | ok |
| 14 | CONHCH₂ | Ph | C | S | 1 | 3 | ok |
| 15 | CH₂ | Ph | C | S | 1 | 3 | ok |
| 16 | CH₂ | Thiophen | C | S | 1 | 2 | ok |
| 17 | CH₂ | Ph | C | S | 1 | 3 | ok |
| 18 | CH₂ | Ph | C | S | 1 | 3 | ok |
| 19 | CH₂ | Ph | C | S | 1 | 3 | ok |
| 20 | CH₂ | Ph | N | N | 1 | 3 | ok |
| 21 | CH₂ | Ph | C | S | 1 | 3 | ok |
| 22 | CH₂ | Ph | C | S | 1 | 3 | ok |
| 23 | CH₂ | Ph | C | S | 1 | 3 | ok |
| 24 | CH₂ | Ph | C | S | 1 | 3 | ok |
| 25 | CH₂ | Ph | C | S | 1 | 3 | ok |
| 26 | CH₂ | Ph | C | S | 1 | 3 | ok |
| 27 | CH₂ | Ph | C | S | 1 | 3 | ok |
| 28 | CH₂ | Ph | C | S | 1 | 3 | ok |
| 29 | CH₂ | Ph | C | S | 1 | 3 | ok |
| 30 | CH₂ | Ph | N | N | 1 | 3 | ok |
| 31 | CH₂ | Ph | N | N | 1 | 2 | ok |

The indication “MS is ok” means that a mass spectrum or HPLC/MS was recorded and the molecular peak M + 1 (MH⁺) and/or M + 18 (MNH₄⁺) and/or M + 23 (MNa⁺) was detected therein. The linkages are indicated in the description of the examples in the experimental part.

The compounds of the formula I are distinguished by beneficial effects on glucose metabolism; in particular, they lower the blood glucose level and are suitable for the treatment of type 1 and type 2 diabetes. The compounds can therefore be employed alone or in combination with other blood glucose-lowering active ingredients (antidiabetics).

The compounds of the formula I are further suitable for the prevention and treatment of late damage from diabetes, such as, for example, nephropathy, retinopathy, neuropathy and syndrome X, obesity, myocardial infarction, peripheral arterial occlusive diseases, thromboses, arteriosclerosis, inflammations, immune diseases, autoimmune diseases such as, for example, AIDS, asthma, osteoporosis, cancer, psoriasis, Alzheimer's, schizophrenia and infectious diseases, with preference for the treatment of type 1 and type 2 diabetes and the prevention and treatment of late damage from diabetes, syndrome X and obesity.

The activity of the compounds was tested as follows:

Preparation of brush border membrane vesicles from the small intestine of rabbits, rats and pigs

Preparation of brush border membrane vesicles from the intestinal cells of the small intestine was carried out by the so-called Mg²⁺ precipitation method. The mucosa of the small intestine was scraped off and suspended in 60 ml of ice-cold Tris/HCl buffer (pH 7.1)/300 mM mannitol, 5 mM EGTA. Dilution to 300 ml with ice-cold distilled water was followed by homogenization with an Ultraturrax (18 shaft, IKA Werk Staufen, FRG) at 75% of the max. power for 2×1 minute, while cooling in ice. After addition of 3 ml of 1M MgCl₂ solution (final concentration 10 mM), the mixture is left to stand at 0° C. for exactly 15 minutes. Addition of Mg²⁺ causes the cell membranes to aggregate and precipitate with the exception of the brush border membranes. After centrifugation at 3 000×g (5 000 rpm, SS-34 rotor) for 15 minutes, the precipitate is discarded and the supernatant, which contains the brush border membranes, is centrifuged at 26 700×g (15 000 rpm, SS-34 rotor) for 30 minutes. The supernatant is discarded, and the precipitate is rehomogenized in 60 ml of 12 mM Tris/HCl buffer (pH 7.1)/60 mM mannitol, 5 mM EGTA using a Potter Elvejhem homogenizer (Braun, Melsungen, 900 rpm, 10 strokes). Addition of 0.1 ml of 1M MgCl₂ solution and incubation at 0° C. for 15 minutes is followed by centrifugation again at 3 000×g for 15 minutes. The supernatant is then centrifuged again at 46 000×g (20 000 rpm, SS-34 rotor) for 30 minutes. The precipitate is taken up in 30 ml of 20 mM Tris/Hepes buffer (pH 7.4)/280 mM mannitol and homogeneously resuspended by 20 strokes in a Potter Elvejhem homogenizer at 1 000 rpm. After centrifugation at 48 000×g (20 000 rpm, SS-34 rotor) for 30 minutes, the precipitate was taken up in 0.5 to 2 ml of Tris/Hepes buffer (pH 7.4)/280 mM mannitol (final concentration 20 mg/ml) and resuspended using a tuberculin syringe with a 27 gauge needle. The vesicles were either used directly after preparation for labeling or transport studies or were stored at −196° C. in 4 mg portions in liquid nitrogen. To prepare brush border membrane vesicles from rat small intestine, 6 to 10 male Wistar rats (bred at Kastengrund, Aventis Pharma) were sacrificed by cervical dislocation, and the small intestines were removed and rinsed with cold isotonic saline. The intestines were cut up and the mucosa was scraped off. The processing to isolate brush border membranes took place as described above. To remove cytoskeletal fractions, the brush border membrane vesicles from rat small intestine were treated with KSCN as chaotropic ion.

To prepare brush border membranes from rabbit small intestine, rabbits were sacrificed by intravenous injection of 0.5 ml of an aqueous solution of 2.5 mg of tetracaine HCl, 100 mg of m-butramide and 25 mg of mebezonium iodide. The small intestines were removed, rinsed with ice-cold physiological saline and frozen in plastic bags under nitrogen at −80° C. and stored for 4 to 12 weeks. For preparation of the membrane vesicles, the frozen intestines were thawed at 30° C. in a water bath and then the mucosa was scraped off. Processing to give membrane vesicles took place as described above.

To prepare brush border membrane vesicles from pig intestine, jejunum segments from a freshly slaughtered pig were rinsed with ice-cold isotonic saline and frozen in plastic bags under nitrogen at −80° C. Preparation of the membrane vesicles took place as described above.

Preparation of brush border membrane vesicles from the renal cortex of the rat kidney

Brush border membrane vesicles were prepared from the cortex of the rat kidney by the method of Biber et al. The kidneys from 6 to 8 rats (200 to 250 g) were removed and the cortex was cut off each kidney as a layer about 1 mm thick. The kidneys were taken up in 30 ml of ice-cold 12 mM Tris/HCl buffer (pH 7.4)/300 mM mannitol and homogenized with an Ultraturrax shaft (level 180 V) for 4×30 seconds while cooling in ice. Addition of 42 ml of ice-cold distilled water was followed by addition of 850 μl of a 1M MgCl₂ solution. Incubation at 0° C. for 15 minutes was followed by centrifugation at 4 500 rpm (Sorvall SS-34 rotor) for 15 minutes. The precipitate was discarded, and the supernatant was centrifuged at 16 000 rpm for 30 minutes. Resuspension of the precipitate in 60 ml of 6 mM Tris/HCl buffer (pH 7.4)/150 mM mannitol/2.5 mM EGTA by 10 strokes in a Potter-Elvejhem homogenizer (900 rpm) and addition of 720 μl of 1 mM MgCl₂ solution was followed by incubation at 0° C. for 15 minutes. The supernatant resulting after centrifugation at 4 500 rpm (SS-34 rotor) for 15 minutes was centrifuged at 16 000 rpm for 30 minutes. The supernatant was homogenized by 10 strokes in 60 ml of 20 mM Tris/Hepes buffer (pH 7.4)/280 mM mannitol, and the resulting suspension was then centrifuged at 20 000 rpm for 30 minutes. The precipitate was resuspended in 20 mM Tris/HCl buffer (pH 7.4)/280 mM mannitol using a tuberculin syringe with a 27 gauge needle and was adjusted to a protein concentration of 20 mg/ml.

Measurement of the glucose uptake by brush border membrane vesicles

The uptake of [¹⁴C]-labeled glucose into brush border membrane vesicles was measured by the membrane filtration method. 10 μl of the brush border membrane vesicle suspension in 10 mM Tris/Hepes buffer (pH 7.4)/300 mM mannitol were added at 30° C. to 90 μl of a solution of 10 μM [¹⁴C]D glucose and the appropriate concentrations of the relevant inhibitors (5-200 μM) in 10 mM Tris/Hepes buffer (pH 7.4)/100 mM NaCl/100 mM [mannitol]. After incubation for 15 seconds, the transport process was stopped by adding 1 ml of ice-cold stop solution (10 mM Tris/Hepes buffer (pH 7.4)/150 mM KCl) and the vesicle suspension was immediately filtered with suction through a cellulose nitrate membrane filter (0.45 μm, 25 mm diameter, Schleicher & Schull) under a vacuum of from 25 to 35 mbar. The filter was washed with 5 ml of ice-cold stop solution. Each measurement was carried out as duplicate or triplicate determination. To measure the uptake of radiolabeled substrates, the membrane filter was dissolved in 4 ml of an appropriate scintillator (Quickszint 361, Zinsser Analytik GmbH, Frankfurt am Main), and the radioactivity was determined by liquid scintillation measurement. The measured values were obtained as dpm (disintegrations per minute) after calibration of the instrument using standard samples and after correction for any chemiluminescence present.

The active ingredients are compared for activity on the basis of IC₅₀ data obtained in the transport assay on rabbit small intestine brush border membrane vesicles for selected substances. (The absolute values may be species- and experiment-dependent.)

Example No. IC₅₀ [μM]

| Example No. | IC₅₀ [μM] |
|---|---|
| Phlorizin | 16 |
| 1 | 4 |
| 2 | 0.4 |
| 3 | 0.3 |

The preparation of various examples is described in detail below, and the other compounds of the formula I were obtained analogously:

Experimental part:

Reaction scheme: synthesis of α-bromoglycosides

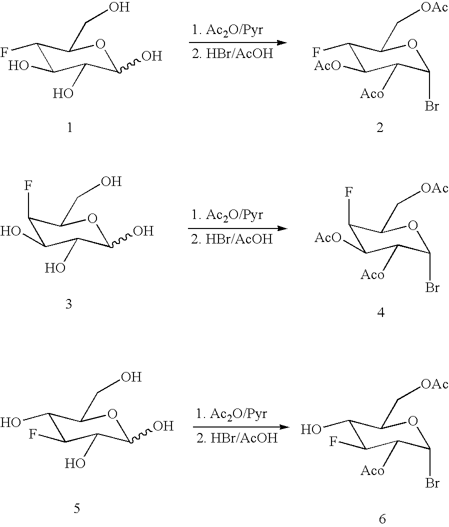

1-Bromo-4-deoxy-4-fluoro-2,3,6-tri-O-acetyl-alpha-D-glucose (2)

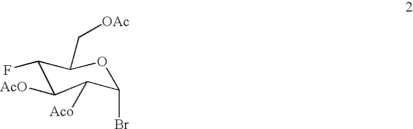

5.0 g (27.5 mmol) of 4-deoxy-4-fluoro-D-glucopyranose 1 (Apollo) are suspended in 50 ml of pyridine and 50 ml of acetic anhydride. The reaction solution is stirred at 45° C. for 4 hours. This results in a clear reaction solution which is concentrated. 12.0 g of crude product are obtained. This crude product is dissolved in 160 ml of 33% strength HBr in glacial acetic acid and left to stand at room temperature for 2 hours. The reaction solution is then poured into a mixture of 300 g of ice and 300 ml of ethyl acetate. The organic phase is washed twice with aqueous NaCl solution, filtered through a little silica gel and concentrated. The residue is separated by chromatography on silica gel (ethyl acetate/heptane=1/1). 8.19 g (80% over 2 stages) of 2 are obtained as a pale yellow solid.

1-Bromo-4-deoxy-4-fluoro-2,3,6-tri-O-acetyl-alpha-D-galactose (4)

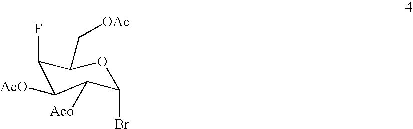

100 mg (0.55 mmol) of 3 are reacted with 3.5 ml of pyridine and 3.5 ml of acetic anhydride in analogy to the preparation of compound 2. 89 mg (44%) of 4 are obtained as an amorphous solid.

1-Bromo-3-deoxy-3-fluoro-2,4,6-tri-O-acetyl-alpha-D-glucose (6)

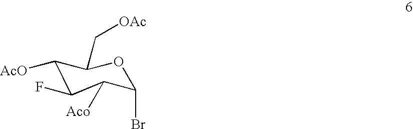

335 mg (1.84 mmol) of 5 are reacted with 10 ml of pyridine and 10 ml of acetic anhydride in analogy to the preparation of compound 2. 628 mg (92%) of 6 are obtained as an amorphous solid.

Reaction scheme: Synthesis of the α-bromoglycoside 10

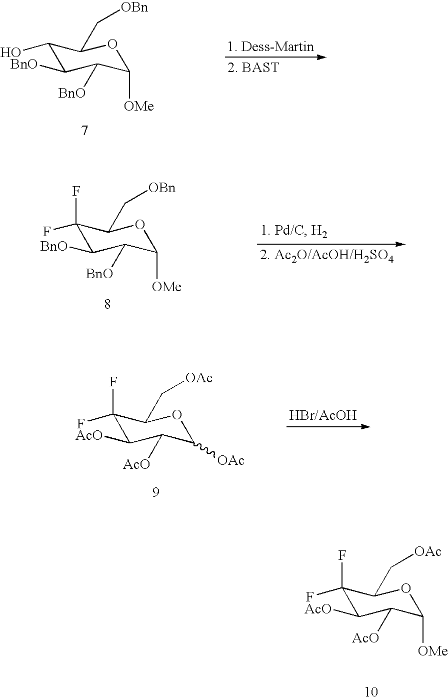

1-Methoxy-4-deoxy-4,4-difluoro-2,3,6-tri-O-benzyl-alpha-D-glucose (8)

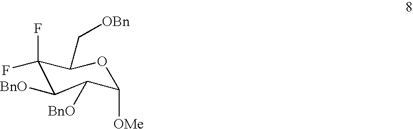

3.69 g (7.9 mmol) of 1-methoxy-2,3,6-tri-O-benzyl-alpha-D-glucose 7 (Tetrahedron Asymmetry 2000, 11, 385-387) were dissolved in 110 ml of methylene chloride and, under an argon atmosphere, 3.6 g (8.5 mmol) of Dess-Martin reagent (Aldrich) are added dropwise. After 3 hours at room temperature, the mixture is diluted with 300 ml of ethyl acetate/n-heptane (1:1) and washed 1× with NaHCO₃ and 1× with Na2S₂O₃ solution. The organic phase is filtered through silica gel and concentrated. The residue is separated by chromatography on silica gel (ethyl acetate/n-heptane 1:1). 2.90 g (79%) of the ketone are obtained. This is dissolved in 30 ml of methylene chloride and, under an argon atmosphere, 4.0 ml of BAST ([bis(2-methoxyethyl)amino]sulfur trifluoride, Aldrich) are added dropwise. After 20 hours at room temperature, the mixture is diluted with 200 ml of ethyl acetate and washed carefully (extensive effervescence) with cold NaHCO₃ solution. The organic phase is filtered through silica gel and concentrated. The residue is separated by chromatography on silica gel (ethyl acetate/n-heptane 1:1). 2.6 g (85%) of 8 are obtained as a colorless oil.

4-Deoxy-4,4-difluoro-1,2,3,6-tetra-O-acetyl-alpha-D-glucose (9)

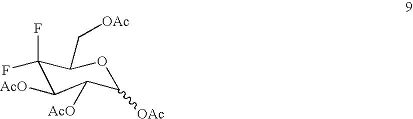

2.30 g (4.7 mmol) of 8 and 2.0 g of Pd/C (10% Pd) are dissolved in 150 ml of methanol and 10 ml of acetic acid and hydrogenated under an atmosphere of 5 bar of hydrogen at room temperature for 16 h. The reaction solution is concentrated and the residue is purified by flash chromatography (methylene chloride/methanol/conc. ammonia, 30/5/1). Yield 850 mg (83%) of 1-methoxy-4-deoxy-4,4-difluoro-alpha-D-glucose as white amorphous solid. C₇H₁₂F₂O₅ (214.17) MS(DCI): 215.4 (M+H⁺).

700 mg (3.3 mmol) of this are dissolved in 3.5 ml of acetic acid and 6.3 ml of acetic anhydride. Addition of 0.2 ml of conc. H₂SO₄ is followed by stirring at 60° C. for 5 h. The reaction solution is then poured into a mixture of 30 g of ice and 30 ml of ethyl acetate. The organic phase is washed twice with aqueous NaCl solution, filtered through a little silica gel and concentrated. The residue is separated by chromatography on silica gel (ethyl acetate/n-heptane 1:1). 300 mg (25%) of 9 are obtained as a mixture of anomers. C₁₄H₁₈F₂O₉ (368.29) MS(DCI): 369.3 (M+H⁺)

1-Bromo-4-dioxy-4,4-difluoro-2,3,6-tri-O-acetyl-alpha-D-glucose (10)

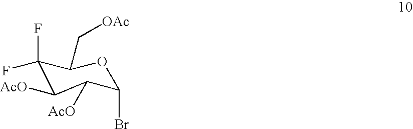

300 mg (0.8 mmol) of tetraacetate 9 are dissolved in 13 ml of 33% strength HBr in glacial acetic acid and left to stand at room temperature for 6 hours. The reaction solution is then poured into a mixture of 10 g of ice and 10 ml of ethyl acetate. The organic phase is washed twice with aqueous NaCl solution, filtered through a little silica gel and concentrated. The residue is separated by chromatography (SiO₂) (ethyl acetate/heptane 1:1). 112 mg (35%) of 10 are obtained as a colorless solid. C₁₂H₁₅BrF₂O₇ (389.15) MS(DCI): 389.2 (M+H⁺).

Reaction scheme: Synthesis of the α-bromoglycosides 14

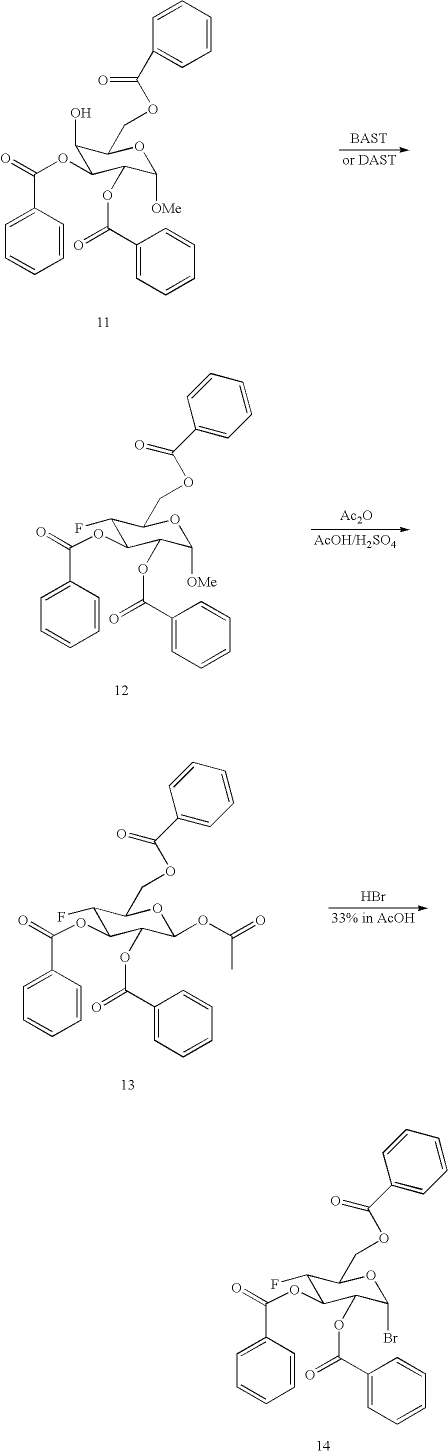

Methyl-2,3,6-tri-O-benzoyl-4-fluoro-4-deoxy-α-D-glucopyranoside (12)

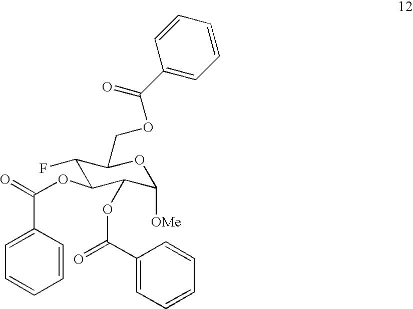

3.0 g of methyl 2,3,6-tri-O-benzoyl-α-D-galactopyranoside (Reist et al., J. Org. Chem 1965, 30, 2312) are introduced into dichloromethane and cooled to −30° C. Then 3.06 ml of [bis(2-methoxyethyl)amino]sulfur trifluoride (BAST) are added dropwise. The reaction solution is warmed to room temperature and stirred for 12 h. The mixture is diluted with dichloromethane, and the organic phase is extracted with H₂O, NaHCO₃ solution and saturated NaCl solution. The organic phase is dried over Na₂SO₄ and concentrated. The crude product is crystallized from ethyl acetate and heptane. 1.95 g of the product 12 are obtained as a colorless solid. C₂₈H₂₅FO₈ (508.51) MS (ESI⁺) 526.18 (M+NH₄⁺). Alternatively, the reaction can also be carried out using 2.8 eq. of diethylaminosulfur trifluoride (DAST); in this case, the reaction solution is refluxed for 18 h after addition. Working up takes place in analogy to the above description.

1-O-Acetyl-2,3,6-tri-O-benzoyl-4-fluoro-4-deoxy-glucose (13)

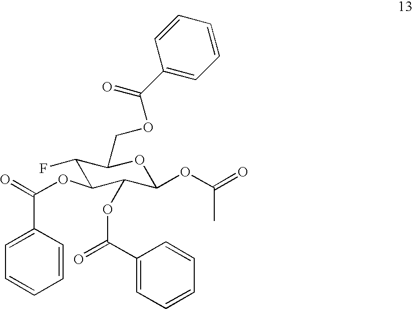

12.0 g of the compound methyl 2,3,6-tri-O-benzoyl-4-fluoro-4-deoxy-α-D-glucopyranoside are suspended in 150 ml of acetic anhydride. 8.4 ml of conc. sulfuric acid are mixed with 150 ml of glacial acetic acid and added to the mixture while cooling in ice. The mixture is stirred at room temperature for 60 h. The reaction mixture is poured into NaHCO₃ solution, and this solution is extracted with chloromethane. The organic phase is washed with NaCl solution, dried with Na₂SO₄ and concentrated. The residue is recrystallized from ethyl acetate and heptane. 5.97 g of the product 13 are obtained as a colorless solid.

C₂₉H₂₅FO₉ (536.52) MS(ESI⁺) 554.15 (M+NH₄⁺).

1-Bromo-4-deoxy-4-fluoro-2,3,6-tri-O-benzoyl-alpha-D-glucose (14)

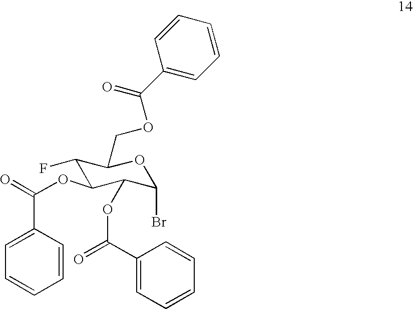

1.44 g of 1-O-acetyl-2,3,6-tri-O-benzoyl-4-fluoro-4-deoxyglucose are dissolved in 20 ml of hydrobromic acid in glacial acetic acid (33%) and stirred at room temperature. After 5 hours, the mixture is added to ice-water, and the aqueous phase is extracted three times with dichloromethane. The collected organic phase is washed with saturated sodium chloride solution, dried over sodium sulfate and evaporated to dryness. The crude product is filtered with ethyl acetate/heptane (70:30) through silica gel. 1.40 g of the product 14 are obtained as a colorless solid.

C₂₇H₂₂BrFO₇ (557.37) MS(ESI⁺) 574.05/576.05 (M+NH₄⁺).

Reaction scheme A: Synthesis of Example 1

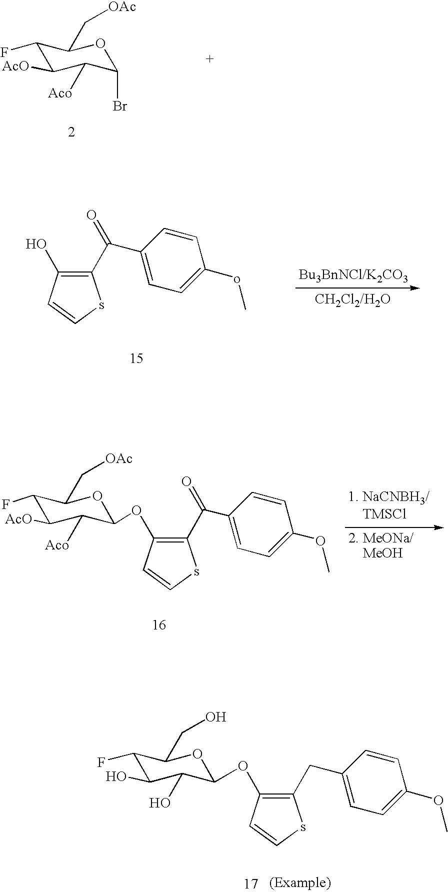

Further exemplary compounds:

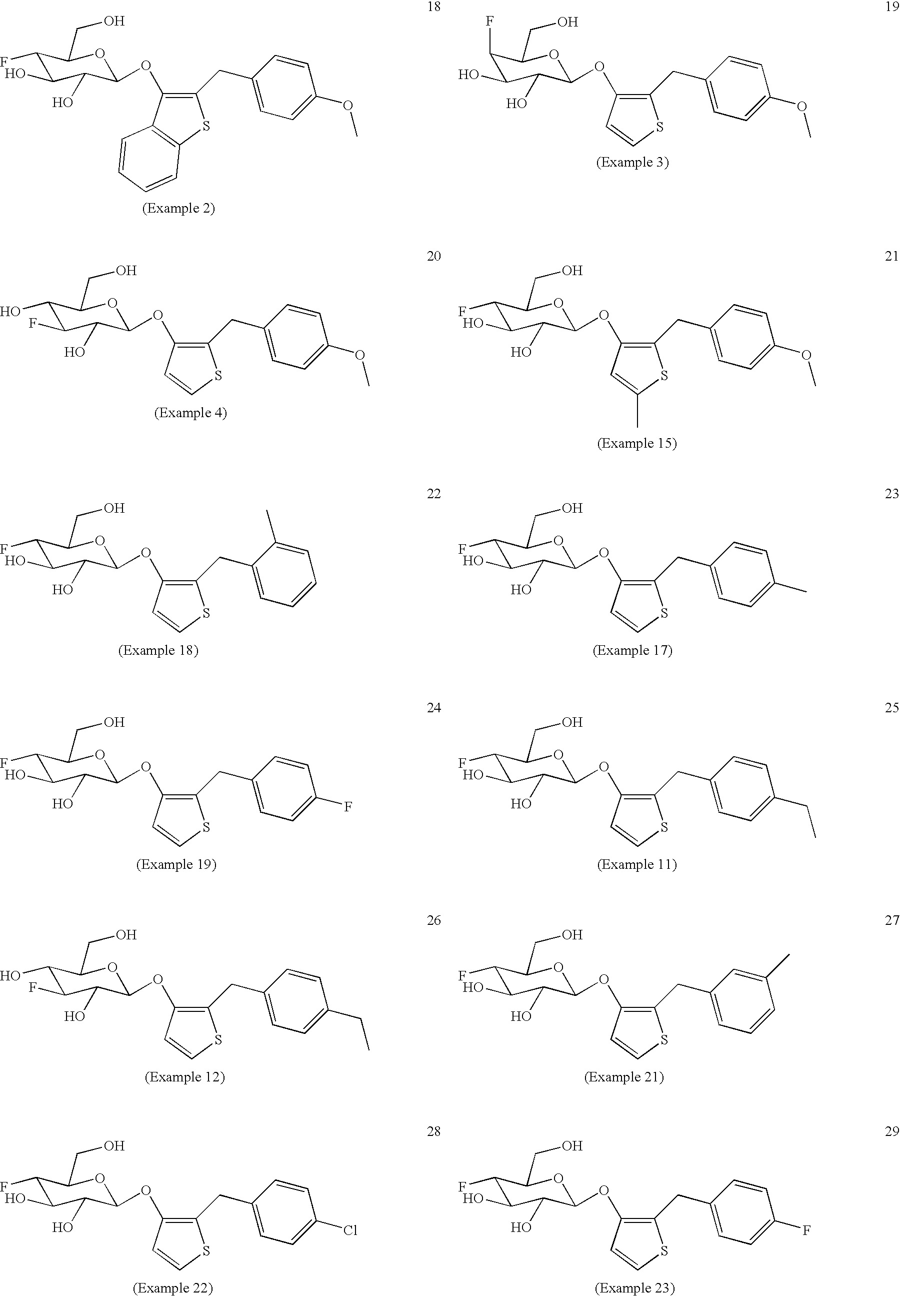

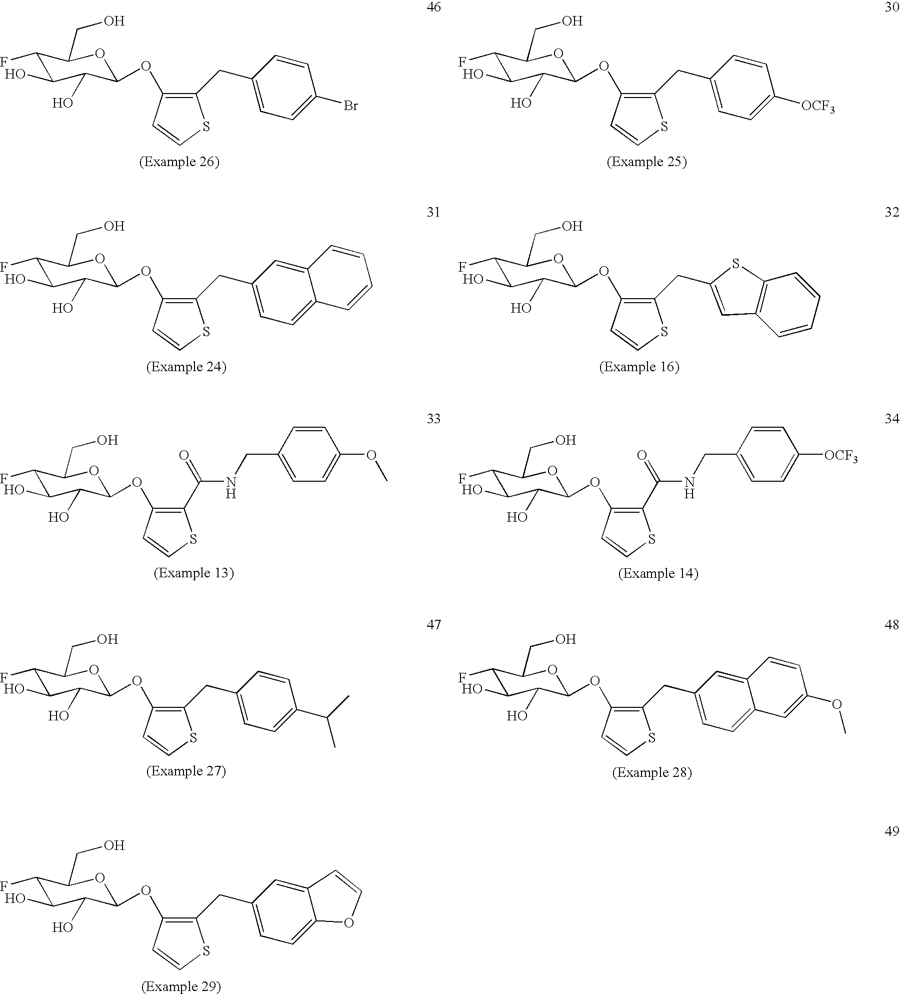

### Example 1

### Compound 17

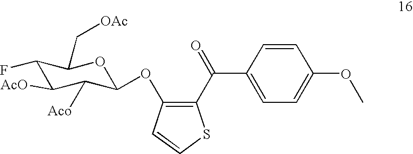

400 mg (1.7 mmol) of (3-hydroxythiophen-2-yl)(4-methoxyphenyl)-methanone 15 LDE Application Number 10231370.9 (2002/0049) and 200 mg (0.54 mmol) of bromide 2 are dissolved in 6 ml of methylene chloride. 160 mg of Bu₃BnNCl (PTC=phase transfer catalyst), 320 mg of K2CO₃ and 0.4 ml of water are successively added to this solution, which is then stirred at room temperature for 20 hours. The reaction solution is diluted with 20 ml of ethyl acetate and filtered through silica gel. The filtrate is concentrated and the residue is separated by chromatography over silica gel (ethyl acetate/heptane=1/1). 160 mg (56%) of 16 are obtained as a colorless solid. C₂₄H₂₅FO₁₀S (524.52) MS(ESI⁺) 525.12 (M+H⁺).

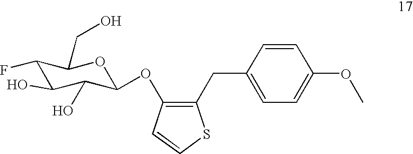

150 mg (0.29 mmol) of compound 16 are dissolved in 4 ml of acetonitrile. This solution is cooled in an ice bath and then 150 mg of NaCNBH₃ and 0.2 ml of TMSCl are added. The cooling is then removed and the mixture is stirred at room temperature for 2 hours. The reaction solution is diluted with 20 ml of ethyl acetate and filtered through silica gel. The filtrate is concentrated, and 150 mg of crude product are obtained. This crude product is taken up in 4 ml of methanol, and 1 ml of 1M NaOMe in MeOH is added. After one hour, the mixture is neutralized with methanolic HCl and concentrated, and the residue is purified by chromatography on silica gel (methylene chloride/methanol/conc. ammonia, 30/5/1). 76 mg (69% over 2 stages) of 17 are obtained as a colorless solid. C₁₈H₂₁FO₆S (384.43) ME(ESI⁺) 403.21 (M+H₂O+H⁺).

### Example 2

### Compound 18

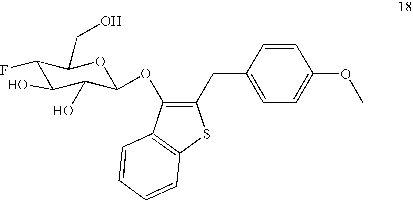

100 mg (0.47 mmol) of (3-hydroxybenzothiophene-2-yl)(4-methoxyphenyl)-methanone (Eur. J. Med. Chem. 1985, 20, 187-189) and 300 mg (0.80 mmol) of bromide 2 are dissolved in 10 ml of chloroform. 120 mg of Bu₃BnNCl (PTC=phase-transfer catalyst) and 1.5 ml of 1 N aqueous sodium hydroxide solution are successively added to this solution, which is then boiled under reflux for 4 hours. The reaction solution is diluted with 20 ml of ethyl acetate and filtered through silica gel. The filtrate is concentrated and the residue is separated by chromatography on silica gel (ethyl acetate/heptane=1/1). 135 mg (51%) of pale yellow solid are obtained. This is converted into compound 18 with 100 mg of NaCNBH₃ and 0.2 ml of TMSCl and then with NaOMe/MeOH in analogy to the preparation of compound 17. 46 mg of 18 are obtained. C₂₂H₂₃FO₆S (434.49) MS(ESI⁻) 479.18 (M+CHO₂⁻).

### Example 3

### Compound 19

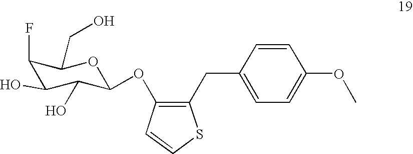

178 mg of (3-hydroxythiophen-2-yl)(4-methoxyphenyl)methanone (15) and 90 mg of bromide 4 are reacted in analogy to the synthesis of example 1, and 49 mg of 19 are obtained as a colorless solid. C₁₈H₂₁FO₆S (384.43) MS(ESI⁺) 403.21 (M+H₂O+H⁺).

### Example 4

### Compound 20

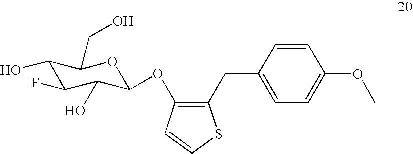

200 mg of (3-hydroxythiophen-2-yl)(4-methoxyphenyl)methanone 15 and 100 mg of bromide 6 are reacted in analogy to the synthesis of example 1, and 59 mg of 20 are obtained as a colorless solid. C₁₈H₂₁FO₆S (384.43) MS(ESI⁺) 403.21 (M+H₂O+H⁺).

Examples 11 (compound 25) and 15 (compound 21) are synthesized in analogy to the synthesis of example 1 starting from the appropriate hydroxythiophenes and the bromide 2.

Examples 16 (compound 32), 17 (compound 23), 18 (compound 22), 19 (compound 24), 21 (compound 27), 22 (compound 28), 23 (compound 29), 24 (compound 31), 25 (compound 30), 26 (compound 46), 27 (compound 47), 28 (compound 48) and 29 (compound 49) are synthesized in analogy to the synthesis of example 1 starting from appropriate hydroxythiophenes and the bromide 14.

Example 12 (compound 26) is synthesized in analogy to the synthesis of example 4 starting from the appropriate hydroxythiophene and bromide 6.

Examples 13 (compound 33) and 14 (compound 34) are synthesized in analogy to the synthesis of compound 16 by reacting the appropriate hydroxythiophenes with the bromide 2 and subsequently deprotecting with NaOMe/MeOH in analogy to example 1.

Example 20 (compound 35) is synthesized in analogy to the synthesis of example 1 starting from hydroxythiophene 15 and the bromide 10.

Reaction scheme B: Synthesis of Example 5

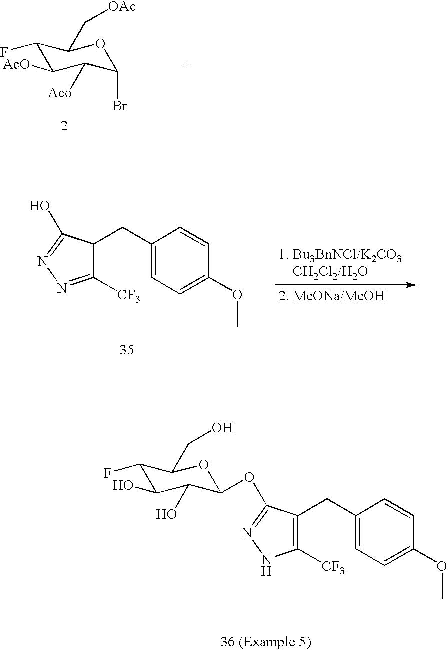

Further exemplary compounds:

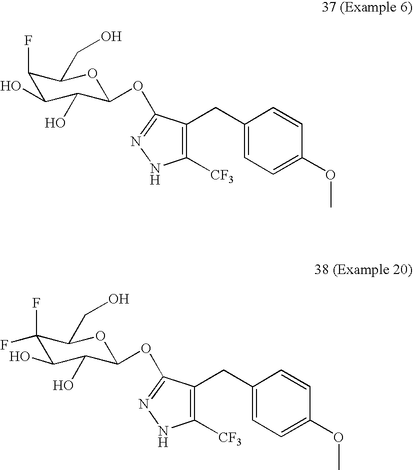

### Example 5

### Compound 36

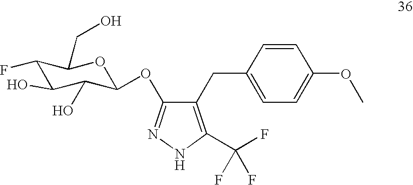

200 mg of 4-(4-methoxybenzyl)-5-methyl-1H-pyrazol-3-ol (35) (J. Med. Chem. 1996, 39, 3920-3928) are glycosylated with 100 mg of bromide 2 in analogy to the synthesis of example 1 and then deprotected with NaOMe/MeOH in analogy to example 1. 49 mg of compound 36 are obtained as a colorless solid. C₁₈H₂₀F₄N₂O₆ (436.36) MS(ESI⁺) 437.21 (M+H⁺).

### Example 6

### Compound 37

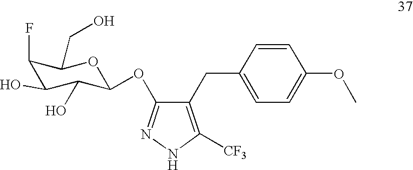

200 mg of 4-(4-methoxybenzyl)-5-methyl-1H-pyrazol-3-ol (35) and 100 mg of bromide 4 are glycosylated in analogy to the synthesis of example 1 and then deprotected with NaOMe/MeOH in analogy to example 1. 89 mg of compound 37 are obtained as a colorless solid. C₁₈H₂₀F₄N₂O₆ (436.36) MS(ESI⁺) 437.21 (M+H⁺).

### Example 20

### Compound 38

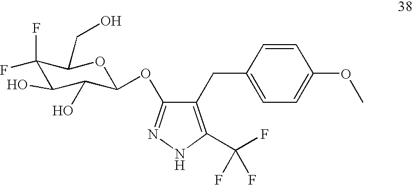

110 mg of 4-(4-methoxybenzyl)-5-methyl-1H-pyrazol-3-ol (35) and 60 mg of bromide 10 are glycosylated in analogy to the synthesis of example 1 and then deprotected with NaOMe/MeOH in analogy to example 1. 49 mg of the compound 38 are obtained as a colorless solid. C₁₈H₁₉F₅N₂O₆ (454.35) MS(ESI⁺) 455.22 (M+H⁺).

Reaction scheme C: Synthesis of Example 8 and Example 10

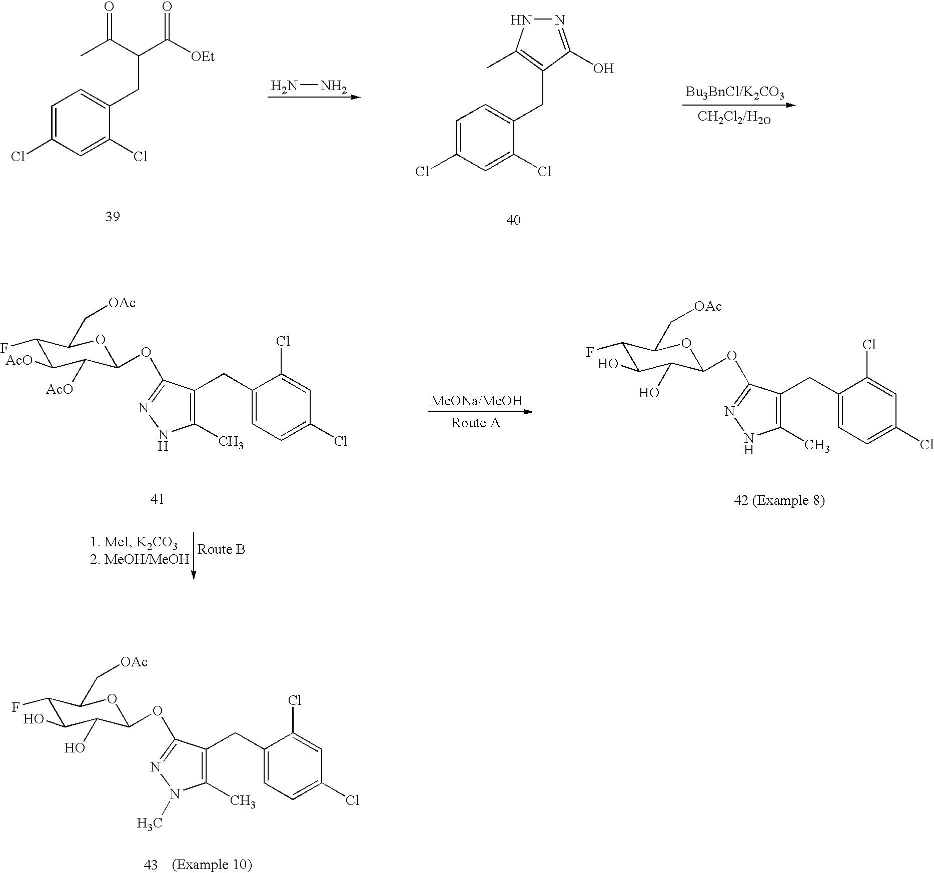

Further exemplary compounds:

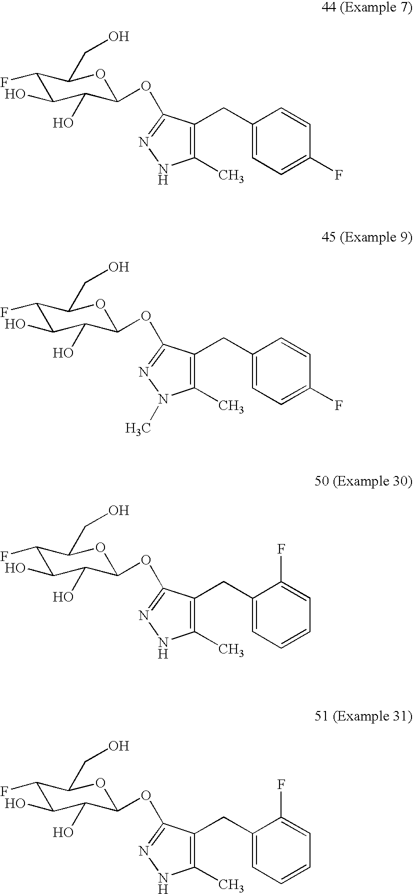

### Example 8

### Compound 42

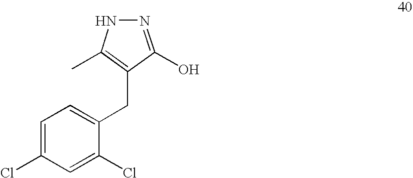

500 mg (1.73 mmol) of ethyl 2-(2,4-dichlorobenzyl)-3-oxobutyrate (39) (Bionet) are boiled with 0.21 ml of 51% pure hydrazine hydrate (3.46 mmol) in 15 ml of toluene with a water trap for 1.5 h. After cooling, the solid is filtered off with suction and washed with toluene and ether. 400 mg (90%) of the compound 40 are obtained as a voluminous white precipitate. C₁₁H₁₀C₁₂N₂O (257.12) MS(ESI): 257 (M+H⁺).

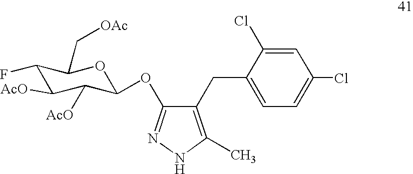

270 mg (1.05 mmol) of 4-(2,4-dichlorobenzyl)-5-methyl-1H-pyrazol-3-ol (40) were dissolved in 25 ml of methylene chloride, and 0.7 ml of water, 1.2 g (8.68 mmol) of potassium carbonate, 84 mg (0.31 mmol) of benzyltriethylammonium bromide and 428 mg (1.15 mmol) of bromide 2 were added, and the mixture was stirred at RT for 18 h. The reaction solution was diluted with methylene chloride and washed once each with water and saturated brine, dried over MgSO₄ and concentrated. The crude product was purified on silica gel. 122 mg (21%) of the compound 41 are obtained as white solid. C₂₃H₂₅Cl₂FN₂O₈ (547.37) MS(ESI): 547 (M+H⁺).

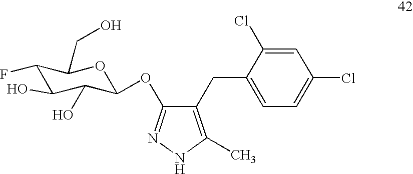

70 mg of (0.1278 mmol) of the compound 41 are dissolved in accordance with route A in 2 ml of methanol, and 1.02 ml (0.511 mmol) of sodium methanolate solution (0.5M) in tetrahydrofuran are added. After 5 min, 27.6 mg (0.516 mmol) of ammonium chloride and 2.0 g of SiO₂ are added. The solution is concentrated and the product is filtered through silica gel and washed first with EtOAc and then with EtOAc/methanol 20:1. 50 mg (90%) of the compound 42 are obtained as a colorless solid.

C₁₇H₁₉Cl₂FN₂O₅ (421.26) MS(ESI): 420 (M+H⁺).

### Example 10

### Compound 43

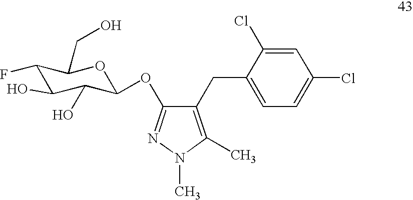

50 mg of compound 41 are dissolved in accordance with route B in 2.0 ml of DMF and, at room temperature, 50 mg of K2CO₃ and 57 μl of methyl iodide are added. After 14 days, 30 ml of EtOAc are added, and the organic phase is washed twice with 20 ml of H₂O each time and concentrated. The crude product is purified by column chromatography (EtOAc/heptane=3:1) and reacted with NaOMe/MeOh in analogy to the preparation of compound 42. 9.1 mg of compound 43 are obtained as a colorless wax. C₁₈H₂₁C₁₂FN₂O₅ (435.24) MS(ESI): 434 (M+H⁺).

Examples 7 (compound 44), 30 (compound 50) and 31 (compound 51) are synthesized in analogy to the synthesis described for example 8 (compound 42) starting from the appropriate β-keto esters.

Example 9 (compound 45) is synthesized in analogy to the synthesis described for example 10 (compound 43) starting from the appropriate β-keto ester.

We claim:

1. A compound of formula I 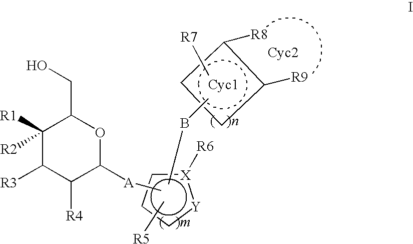
wherein
R1 and R2 are each independently F or H or one of said radicals R1 and R2 may be OH;
R3 is OH or F, with the proviso that at least one of the radicals R1, R2 and R3 must be F;
R4 is OH;
A is O, NH, CH₂, S or a bond;
X is C, O, S or N, with the proviso that X is C when Y is O or S;
Y is N, O or S;
m is 1 or 2;
R5 is hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl, (C₁-C₆)-alkoxycarboxyl,
wherein said CO(C₁-C₆)-alkyl, COO(C₁-C₆)-alkyl, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl and (C₁-C₆)— alkoxycarboxyl radicals are optionally substituted with one or more fluorine atoms,
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl,
wherein said SO₂NH(C₁-C₆)-alkyl, SO₂N[(C₁-C₆)-alkyl]2, S—(C₁-C₆)-alkyl, SO—(C₁-C₆)-alkyl and SO₂—(C₁-C₆)-alkyl radicals are optionally substituted with one or more fluorine atoms, and wherein the phenyl ring of said S—(CH₂)ₒ-phenyl, SO—(CH₂)ₒ-phenyl and SO₂—(CH₂)ₒ-phenyl radicals is optionally mono- or disubstituted with F, Cl, Br, OH, CF₃, NO₂, CN, OCF₃, O—(C₁-C₆)-alkyl, (C₁-C₆)-alkyl or NH₂, and wherein o is 0, 1, 2, 3, 4, 5, or 6,
NH₂, NH—(C₁-C₆)-alkyl, N((C₁-C₆)-alkyl)₂, NH(C₁-C₇)-acyl, phenyl or O—(CH₂)ₒ-phenyl,
wherein the phenyl ring of said phenyl and O—(CH₂)ₒ-phenyl radicals is optionally mono-, di-, or trisubstituted with F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂, and wherein o is as hereinabove defined;
or, when Y is S, R5 and R6 taken together with the carbon atoms to which they are attached may form a phenyl ring;
R6 is H, (C₁-C₆)-alkyl, (C₁-C₆)-alkenyl, (C₃-C₆)-cycloalkyl, or phenyl wherein said phenyl radical is optionally substituted with halogen or (C₁-C₄)-alkyl;
B is (C₀-C₁₅)-alkanediyl, wherein one or more of the carbon atoms in said alkanediyl radical may be replaced, independently of one another, with —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n is 0, 1, 2, 3 or 4;
Cyc1 is a 3-, 4-, 5-, 6- or 7-membered saturated, partially saturated or unsaturated ring, wherein one carbon atom of said ring may be replaced by O, N or S;
R7, R8, and R9 are each independently hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl,
wherein said COO(C₁-C₆)-alkyl, CO(C₁-C₄)-alkyl, CON H(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl and (C₁-C₆)-alkyl-O—(C₁-C₆)-alkyl radicals are optionally substituted with one or more fluorine atoms,
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SCF₃, SO—(C₁-C₆)-alkyl, SO—(CH₂)ₒ-phenyl, SO₂—(C₁-C₆)-alkyl, SO₂—(CH₂)ₒ-phenyl,
wherein said SO₂NH(C₁-C₆)-alkyl, SO₂N[(C₁-C₆)-alkyl]₂, S—(C₁-C₆)-alkyl, SO—(C₁-C₆)-alkyl and SO₂—(C₁-C₆)-alkyl radicals are optionally substituted with one or more fluorine atoms, and wherein the phenyl ring of said S—(CH₂)ₒ-phenyl, SO—(CH₂)ₒ-phenyl and SO₂—(CH₂)ₒ-phenyl radicals is optionally mono- or disubstituted with F, Cl, Br, OH, CF₃, NO₂, CN, OCF₃, O—(C₁-C₆)-alkyl, (C₁-C₆)-alkyl or NH₂, and wherein o is as hereinabove defined,
NH₂, NH—(C₁-C₆)-alkyl, N((C₁-C₆)-alkyl)₂, NH(C₁-C₇)-acyl, phenyl or O—(CH₂)ₒ-phenyl,
wherein the phenyl ring of said phenyl and O—(CH₂)ₒ-phenyl radicals is optionally mono-, di-, or trisubstituted with F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO—(C₁-C₆)-alkyl or CONH₂, and wherein o is as hereinabove defined;
or R8 and R9 taken together with the carbon atoms to which they are attached form a 5-, 6- or 7-membered, saturated, partially saturated or completely unsaturated ring herein referred to as Cyc2,
wherein one or two carbon atom(s) in said Cyc2 ring are optionally replaced by N, O or S, and wherein said Cyc2 ring is optionally substituted with (C₁-C₆)-alkyl, (C₂-C₅)— alkenyl or (C₂-C₅)-alkynyl,
wherein said (C₁-C₆)-alkyl, (C₂-C₅)-alkenyl and (C₂-C₅)-alkynyl radicals are optionally substituted with F, Cl, OH, CF₃, NO₂, CN, COO(C₁-C₄)-alkyl, CONH₂, CONH(C₁-C₄)-alkyl or OCF₃, and wherein a —CH₂— group contained in said (C₁-C₆)-alkyl, (C₂-C₅)-alkenyl and (C₂-C₅)-alkynyl radicals is optionally replaced by —O—;
and pharmaceutically acceptable salts thereof.

2. The compound of claim 1 wherein:
R1 and R2 are each independently F or H or one of said radicals R1 and R2 may be OH,
with the proviso that at least one of said radicals R1 and R2 is F;
R3 is OH;
R4 is OH;
A is O or NH;
X is C, O or N, with the proviso that X is C when Y is S;
Y is N or S;
m is 1 or 2;
R5 is hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl or (C₁-C₆)— alkoxycarboxyl,
wherein said CO(C₁-C₆)-alkyl, COO(C₁-C₆)-alkyl, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, (C₁-C₆)— alkoxycarboxyl and SO—(C₁-C₆)-alkyl radicals are optionally substituted with one or more fluorine atoms,
or when Y is S, R5 and R6 taken together with the carbon atoms to which they are attached may form a phenyl ring;
R6 is H, (C₁-C₆)-alkyl, (C₁-C₆)-alkenyl, (C₃-C₆)-cycloalkyl, or phenyl wherein said phenyl radical is optionally substituted with halogen or (C₁-C₄)-alkyl;
B is (C₀-C₁₅)-alkanediyl, wherein one or more of the carbon atoms in said alkanediyl radical may be replaced, independently of one another, with —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n is 0, 1, 2, 3 or 4;
Cyc1 is a 3-, 4-, 5-, 6- or 7-membered saturated, partially saturated or unsaturated ring, wherein one carbon atom of said ring may be replaced by O or S;
R7, R8, and R9 are each independently hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, S—(C₁-C₆)-alkyl, CF₃ or SO—(C₁-C₆)-alkyl,
wherein said COO(C₁-C₆)-alkyl, CO(C₁-C₄)-alkyl, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, S—(C₁-C₆)-alkyl and SO—(C₁-C₆)-alkyl radicals are optionally substituted with one or more fluorine atoms,
or R8 and R9 taken together with the carbon atoms to which they are attached form a 5-, 6- or 7-membered, saturated, partially saturated or completely unsaturated ring herein referred to as Cyc2,
wherein one or two carbon atom(s) in said Cyc2 ring is optionally replaced by N, O or S, and wherein said Cyc2 ring is optionally substituted with (C₁-C₆)-alkyl, (C₂-C₅)— alkenyl or (C₂-C₅)-alkynyl,
wherein said (C₁-C₆)-alkyl, (C₂-C₅)-alkenyl and (C₂-C₅)-alkynyl radicals are optionally substituted with F, Cl, OH, CF₃, NO₂, CN, COO(C₁-C₄)-alkyl, CONH₂, CONH(C₁-C₄)-alkyl or OCF₃, and wherein a —CH₂— group contained in said (C₁-C₆)-alkyl, (C₂-C₅)-alkenyl and (C₂-C₅)-alkynyl radicals is optionally replaced by —O—.

3. The compound of claim 1 wherein the sugar residues are beta(β)-linked and the stereochemistry in the 2, 3 and 5 position of the sugar residue has the D-gluco configuration.

4. The compound of claim 1 wherein:
R1 and R2 are each independently F or H or one of said radicals R1 and R2 may be OH,
with the proviso that at least one of said radicals R1 and R2 is F;
R3 is OH;
R4 is OH;
A is O;
X is C, O or N, with the proviso that X is C when Y is S;
Y is N or S;
m is 1;
R5 is hydrogen, F, Cl, CF₃, OCF₃, COO(C₁-C₄)-alkyl, (C₁-C₅)-alkyl, (C₂-C₄)-alkenyl, (C₂-C₄)-alkynyl, (C₁-C₄)-alkoxy, HO—(C₁-C₄)-alkyl, (C₁-C₄)-alkyl-O—(C₁-C₄)-alkyl, phenyl, benzyl, (C₁-C₄)-alkoxycarboxyl, OCH₂CF₃ or (C₁-C₄)-alkyl-CF₂—,
or when Y is S, R5 and R6 taken together with the carbon atoms to which they are attached may form a phenyl ring;
R6 is H, (C₁-C₆)-alkyl, (C₁-C₆)-alkenyl, (C₃-C₆)-cycloalkyl, or phenyl wherein said phenyl radical is optionally substituted with halogen or (C₁-C₄)-alkyl;
B is (C₁-C₄)-alkanediyl, wherein one carbon atom in said alkanediyl radical may be replaced with —O—, —(C═O)—, —CH(OH)—, —CHF—, —CF₂—, —CO—NH—;
n is 2 or 3;
Cyc1 is an unsaturated 5- or 6-membered ring, wherein one carbon atom of said ring may be replaced by O or S;
R7, R8, and R9 are each independently hydrogen, F, Cl, Br, I, OH, (C₁-C₄)-alkyl, OCH₂CF₃, (C₁-C₈)-alkoxy, HO—(C₁-C₆)-alkyl, (C₁-C₄)— alkyl-O—(C₁-C₄)-alkyl, S—(C₁-C₄)-alkyl, SCF₃ or OCF₃,
or R8 and R9 taken together form the radicals —C═CH—O—, —CH═CH—S— or —CH═CH—CH═CH— and, with the carbon atoms to which they are attached, form an unsaturated or partially saturated 5- or 6-membered ring, said ring being optionally substituted by (C₁-C₄)-alkoxy or —O—(CH₂)ₚ—O— wherein p is 1 or 2.

5. The compound of claim 1 wherein:
R1 and R2 are each independently F or H,
with the proviso that at least one of said radicals R1 and R2 is F;
R3 is OH;
R4 is OH;
A is O;
X is C and Y is S, or
is 0 and Y is N, or
is N and Y is N;
m is 1;
R5 is hydrogen, CF₃, (C₁-C₆)-alkyl, or when Y is S, R5 and R6 taken together with the carbon atoms to which they are attached may form a phenyl ring,
R6 is H, (C₁-C₄)-alkyl or phenyl;
B is —CH₂—, —C₂H₄—, —C₃H₆—, —CO—NH—CH₂— or —CO—CH₂—CH₂—;
n is 2 or 3;
Cyc1 is an unsaturated 5- or 6-membered ring, wherein one carbon atom of said ring may be replaced by S;
R7, R8, and R9 are each independently hydrogen, F, Cl, Br, I, (C₁-C₆)-alkyl, (C₁-C₄)-alkoxy, S—(C₁-C₄)-alkyl, SCF3 or OCF3,
or R8 and R9 taken together form the radicals —C═CH—O— or —CH═CH—CH═CH— and, with the carbon atoms to which they are attached, form an unsaturated or partially saturated 5- or 6-membered ring, said ring being optionally substituted by (C₁-C₄)-alkoxy.

6. The compound of claim 1 wherein:
R1 and R2 are each independently F or H, with the proviso that at least one of said radicals R1 and R2 is F;
R3 is OH;
R4 is OH;
A is O;
X is C and Y is S, or
is N and Y is N;
m is 1;
R5 is hydrogen, CF₃, (C₁-C₆)-alkyl, or when Y is S, R5 and R6 taken together with the carbon atoms to which they are attached may form a phenyl ring,
R6 is H or (C₁-C₄)-alkyl;
B is —CH₂— or —CO—NH—CH₂—;
n is 2 or 3;
Cyc1 is phenyl or thiophene;
R7, R8, and R9 are each independently hydrogen or Cl,
or R8 and R9 taken together with the carbon atoms to which they are attached, form a furan ring or a phenyl ring optionally substituted with methoxy.

7. A pharmaceutical composition comprising a compound of claim 1 and a pharmaceutically acceptable carrier.

8. A pharmaceutical composition comprising a compound of claim 1 and one or more blood glucose-lowering active ingredients.

9. A method of treating type 1 or type 2 diabetes which comprises administering to a patient in need thereof a therapeutically effective amount of a compound of claim 1.

10. A method of lowering blood glucose which comprises administering to a patient in need thereof a therapeutically effective amount of a compound of claim 1.

11. A method of treating type 1 or type 2 diabetes which comprises administering to a patient in need thereof a therapeutically effective amount of a compound of claim 1 with at least one other blood glucose-lowering active ingredient.

12. A method of lowering blood glucose which comprises administering to a patient in need thereof a therapeutically effective amount of a compound of claim 1 with at least one other blood glucose-lowering active ingredient.
